(19) United States
(12) Patent Application Publication
(10) Pub. No.: US 2023/0043385 A1
(43) Pub. Date: Feb. 9, 2023

(54) INTRAVASCULAR BLOOD PUMPS, MOTORS, AND FLUID CONTROL

(71) Applicant: SHIFAMED HOLDINGS, LLC, Campbell, CA (US)

(72) Inventors: Daniel VARGHAI, Campbell, CA (US); Ari RYAN, Sunnyvale, CA (US)

(73) Assignee: SHIFAMED HOLDINGS, LLC, Campbell, CA (US)

(21) Appl. No.: 17/794,002

(22) PCT Filed: Feb. 5, 2021
(86) PCT No.: PCT/US2021/016888

§ 371 Date: Jul. 20, 2022

Related U.S. Application Data
(60) Provisional application No. 62/970,460, filed on Feb. 5, 2020.

(51) Int. Cl. A61M 60/13; A61M 60/221

(52) U.S. Cl. CPC A61M 60/13; A61M 60/221

(57) ABSTRACT

Intravascular blood pumps systems and methods of use. The blood pump system includes a catheter portion, a pump portion, and a motor. The motors may be isolated from a return fluid pathway, and may be adapted to rotate about an axis that is spaced from a rotational axis of a drive shaft.

### CROSS REFERENCE TO RELATED APPLICATIONS

This application claims priority to U.S. Provisional Application No. 62/970,460, filed on Feb. 5, 2020, which is incorporated herein by reference in its entirety for all purposes.

This application may be related to International Patent Application No. PCT/US2019/055038, filed on Oct. 7, 2019, published as WO 2020/073047, and entitled “INTRAVASCULAR BLOOD PUMPS AND METHODS OF USE”, which is incorporated herein by reference in its entirety. Additionally, the disclosure from any of the following references may be incorporated by reference herein for all purposes: U.S. Pat. Nos. 9,675,739; 5,964,694; 7,027,875; 9,789,238; and 8,814,933.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. There is a need for ways to improve the volume or stability of cardiac outflow for these patients, especially during corrective procedures.

Intra-aortic balloon pumps (IABP) are commonly used to support circulatory function, such as treating heart failure patients. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

An IABP commonly works by being placed within the aorta and being inflated and deflated in counterpulsation fashion with the heart contractions, and one of the functions is to attempt to provide additive support to the circulatory system.

More recently, minimally invasive rotary blood pumps have been developed that can be inserted into the body in connection with the cardiovascular system, such as pumping arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient.

The smallest rotary blood pumps currently available can be percutaneously inserted into the vasculature of a patient through an access sheath, thereby not requiring surgical intervention, or through a vascular access graft. A description of this type of device is a percutaneously inserted ventricular support device.

There is a need to provide additional improvements to the field of ventricular support devices and similar blood pumps for treating compromised cardiac blood flow. There is also a need for motor assemblies that facilitate rotation of one or more impellers of the blood pumps.

### SUMMARY OF THE DISCLOSURE

The present disclosure relates to fluid movement devices, such as intravascular blood pumps, and their methods of use. The present disclosure is also related to motors and motor assemblies adapted to drive the rotation of one or more impellers of an intravascular blood pump.

One aspect of the disclosure is a blood pump system, which may also be referred to herein as a catheter blood pump.

In this aspect, the blood pump system may include a motor that is coupled to and in rotational communication with a mechanical driving member, the mechanical driving member rotatable about a first axis, the mechanical driving member mechanically coupled to a mechanical driven member rotatable about a second axis that is spaced from the first axis, wherein rotation of the mechanical driving member by the motor causes rotation of the mechanical driven member about the second axis.

In this aspect, the mechanical driven member may be coupled to a rotatable first magnetic assembly (optionally an outer magnetic assembly) that is rotatable about the second axis such that rotation of the mechanical driven member causes rotation of the first magnetic assembly. A rotatable second magnetic assembly (optionally an inner magnetic assembly) may be magnetically coupled with the first magnetic assembly such that rotation of the first magnetic assembly causes rotation of the second magnetic assembly about the second axis.

In this aspect, a fluid return pathway may include a volume that is between the first and second magnetic assemblies, wherein the fluid return pathway may be in fluid communication with a fluid outlet.

In this aspect, a stationary return fluid member may be disposed around a rotatable inner magnetic assembly.

In this aspect, the second magnetic assembly may be in rotational communication (direct or indirect) with a proximal region of a drive shaft such that rotation of the second magnetic assembly causes rotation of the drive shaft about the second axis, and the drive shaft may be in rotational communication with one or more blood pump impellers.

In this aspect, the system may include a purge fluid inlet, optionally that is distal to the mechanical driven member. The fluid inlet may be in communication with a clean purge pathway within a catheter that extends distally relative to the motor, wherein the catheter may extend to or proximate the pump portion.

In this aspect, a return fluid outlet may be proximal to the mechanical driven member, the return fluid outlet in communication with a return fluid pathway.

In this aspect, the return fluid pathway may include a lumen in the catheter and a volume created by and between the stationary return fluid member and a rotatable inner magnetic assembly.

In this aspect, a rotatable first magnetic assembly may be coupled to an inner surface of a mechanical driven member. In this aspect, a rotatable first magnetic assembly may be coupled to an outer surface of the mechanical driven member.

In this aspect, a mechanical coupling between a mechanical driving member and a mechanical driven member may include a geared mechanical coupling, such as a plurality of gears with teeth.

In this aspect, a mechanical coupling between a mechanical driving member and a mechanical driven member may include a friction gear, optionally adapted to modify a force applied on the driven member by the driving member.

In this aspect, the system may include a housing in which at least a portion of the motor is disposed, wherein the housing may be sized and configured to maintain the first and second axes at a fixed distance.

In this aspect, the system may further comprise a housing in which at least a portion of the motor is disposed, wherein the housing may be sized and configured to allow a distance between the first and second axes to be controllably adjusted.

In this aspect, the system may further comprise a housing in which at least a portion of the motor is disposed, wherein the housing may be sized and configured to allow adjustable modification to one or more of a degree of contact between a driving and driven member or a force applied on the driven member by the driving member.

In this aspect, a driving member may have a diameter that is larger than a diameter of a driven member.

In this aspect, a speed multiplication between a driving and driven member may be greater than 1:1, optionally about 1.5:1, and optionally about 2:1, and optionally between 1:1 and 3:1. The speed multiplication may help the motor spin at a lower speed, which may help reduce motor vibrations.

In this aspect, a driven member may have an outer surface that interfaces with a surface of the driving member, the outer surface of the driven member may have a smaller diameter than at least a portion of the outer magnetic assembly, such as in FIGS. 13 and 14A-14D.

One aspect of this disclosure is a system include a housing including a first portion and a second portion, the first portion sized and configured to receive at least a portion of a motor therein, wherein the housing is adapted to maintain at least one of force (e.g., a constant force) or contact on the driven member by the driving member in the event of wear from at least one of the driving member or the driven member from use.

In this aspect, the housing may include one or more force maintainers that are adapted to maintain the at least one of force or contact. Force maintainers may comprise a screw and a spring, wherein the first and second portions may each include a threaded aperture therein to receive the screw, optionally wherein a spring constant maintains the at least one or force or contact.

In this aspect, the system may include a motor that is coupled to and in rotational communication with a mechanical driving member, the mechanical driving member rotatable about a first axis, the mechanical driving member mechanically coupled to a mechanical driven member rotatable about a second axis that is spaced from the first axis, wherein rotation of the mechanical driving member by the motor causes rotation of the mechanical driven member about the second axis.

In this aspect, the mechanical driven member may be coupled to a rotatable first magnetic assembly (optionally outer magnetic assembly) that is rotatable about the second axis such that rotation of the mechanical driven member causes rotation of the first magnetic assembly.

In this aspect, the system may include a rotatable second magnetic assembly (optionally an inner magnetic assembly) that is magnetically coupled with the first magnetic assembly such that rotation of the first magnetic assembly causes rotation of the second magnetic assembly about the second axis.

In this aspect, a second magnetic assembly may be in rotational communication with a proximal end of a drive shaft (directly or indirectly) such that rotation of the second magnetic assembly causes rotation of the drive shaft about the second axis, where the drive shaft is in rotational communication with a blood pump impeller.

In this aspect, the housing may include a hinge that facilitates relative motion between the first and second portion.

In this aspect, the system may further comprise a case. The case may optionally include a first portion and a second portion movable relative to each other to provide access to a housing receiving area in which the housing may be disposed. First and second case portions may have a closed configuration such that a mechanical driving and driven members of the motor assembly are protected from contact from ambient objects.

One aspect of the disclosure is related to maintaining at least one of force or contact between mechanical driving and driven members, such as any of the mechanical driving and driven members herein. This aspect may include any step or steps described herein related to the maintaining at least one of force or contact therebetween.

### BRIEF DESCRIPTION OF THE DRAWINGS

FIG. 1 is a side view of an exemplary pump portion that includes a conduit, a plurality of impellers, an expandable member

FIG. 2 is a side view of an exemplary pump portion that includes a conduit, a plurality of impellers, and a plurality of expandable members.

FIGS. 3A, 3B, 3C and 3D illustrate an exemplary pump portion that includes a conduit, a plurality of impellers, and a plurality of expandable members.

FIG. 4 illustrates an exemplary placement of a pump portion, the pump portion including a conduit, a plurality of expandable members, and a plurality of impellers.

FIGS. 5A-D illustrates an exemplary blood pump that includes a guidewire pathway and at least one fluid purge pathway.

FIGS. 6A and B illustrates an exemplary blood pump that includes a guidewire pathway and at least two fluid purge pathways that are not in fluid communication.

FIGS. 7A-7F illustrate an exemplary sequence of steps that may be carried out based on an exemplary method of using an exemplary blood pump.

FIG. 8A illustrates at least a portion of an exemplary blood pump system including an intravascular blood pump.

FIG. 8B illustrates an exemplary fluid pathway through at least a portion of the exemplary blood pump system of FIG. 8A.

FIGS. 9A and 9B illustrate an exemplary external motor for an intravascular blood pump: FIG. 9A shows a cross-section view of the motor showing an exemplary fluid pathway therethrough; and FIG. 9B shows an external side view of the motor.

FIG. 10 illustrates a cross-section view of an exemplary external motor for an intravascular blood pump showing an exemplary fluid pathway and possible fluid ingress toward a stator motor component.

FIG. 11 illustrates a cross-section view of an exemplary external motor for an intravascular blood pump showing an exemplary isolation component to prevent ingress of fluid toward a stator motor component.

FIGS. 12A-12C illustrate an exemplary blood pump with a motor that is isolated from a return fluid pathway.

FIG. 13 illustrates an exemplary blood pump with a motor that is isolated from a return fluid pathway.

FIGS. 14A-14D illustrate an exemplary blood pump with a motor that is isolated from a return fluid pathway.

FIGS. 15A and 15B illustrate an exemplary blood pump system including a case adapted to house a motor assembly therein.

### DETAILED DESCRIPTION

The present disclosure is related to medical devices, systems, and methods of use and manufacture. Medical devices herein may include a pump portion adapted and configured to be disposed within a physiologic vessel, wherein the pump includes one or more components that act upon fluid. For example, pump portions herein may include one or more impellers that are configured such that when rotated, they facilitate the movement of a fluid such as blood.

FIG. 1 is a side view illustrating a distal portion of an exemplary intravascular fluid pump, including pump portion 1600, wherein pump portion 1600 includes proximal impeller 1606 and distal impeller 1616, both of which are in operable communication with drive cable 1612. Pump portion 1600 is in an expanded configuration in FIG. 1, but is adapted to be collapsed to a delivery configuration so that it can be delivered with a lower profile. The impellers can in rotational communication with drive cable 1612, directly or indirectly. Drive cable 1612 is in operable communication with an external motor, not shown, and extends through elongate shaft 1610. The phrases “pump portion” and “working portion” (or derivatives thereof) may be used herein interchangeably unless indicated to the contrary. For example without limitation, “pump portion” 1600 can also be referred to herein as a “working portion.”

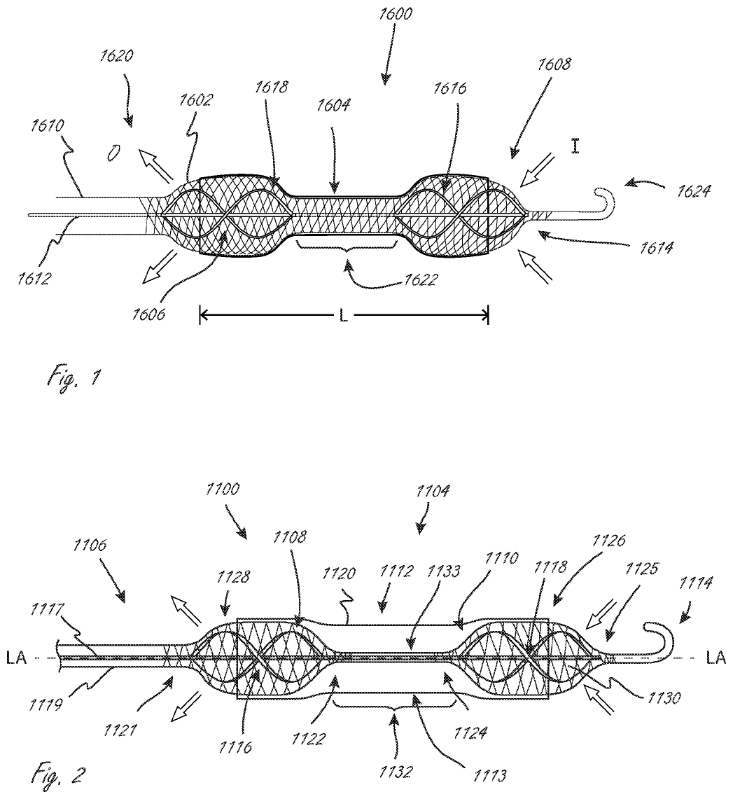

FIG. 2 is a side view illustrating a deployed configuration (shown extracorporally) of a distal portion of an exemplary embodiment of a fluid movement system. Exemplary system 1100 includes pump portion 1104 (which as set forth herein may also be referred to herein as a pump portion) and an elongate portion 1106 extending from pump portion 1104. Elongate portion 1106 can extend to a more proximal region of the system, not shown for clarity, and that can include, for example, a motor. Pump portion 1104 includes first expandable member 1108 and second expandable member 1110, axially spaced apart along a longitudinal axis LA of pump portion 1104. Spaced axially in this context refers to the entire first expandable member being axially spaced from the entire second expandable member along a longitudinal axis LA of pump portion 1104. A first end 1122 of first expandable member 1108 is axially spaced from a first end 1124 of second expandable member 1110. Some “expandable members” herein may also be referred to herein as baskets.

First and second expandable members 1108 and 1110 generally each include a plurality of elongate segments disposed relative to one another to define a plurality of apertures 1130, only one of which is labeled in the second expandable member 1110. The expandable members can have a wide variety of configurations and can be constructed in a wide variety of ways, such as any of the configurations or constructions in, for example without limitation, U.S. Pat. No. 7,841,976, or the tube in 6,533,716, which is described as a self-expanding metal endoprosthetic material. For example, without limitation, one or both of the expandable members can have a braided construction or can be at least partially formed by laser cutting a tubular element.

Pump portion 1104 also includes blood flow conduit 1112, which in this embodiment is supported by first expandable member 1108 and to second expandable member 1110. Conduit 1112 also extends axially in between first expandable member 1108 and second expandable member 1110 in the deployed configuration. A central region 1113 of conduit 1112 spans an axial distance 1132 where the pump portion is void of first and second expandable members 1108 and 1110. Central region 1113 can be considered to be axially in between the expandable members. Distal end 1126 of conduit 1112 does not extend as far distally as a distal end 1125 of second expandable member 1110, and proximal end of conduit 1128 does not extend as far proximally as proximal end 1121 of first expandable member 1108.

When the disclosure herein refers to a conduit being coupled to an expandable member, the term coupled in this context does not require that the conduit be directly attached to the expandable member so that conduit physically contacts the expandable member. Even if not directly attached, however, the term coupled in this context refers to the conduit and the expandable member being joined together such that as the expandable member expands or collapses, the conduit also begins to transition to a different configuration and/or size. Coupled in this context therefore refers to conduits that will move when the expandable member to which it is coupled transitions between expanded and collapsed configurations. The conduits herein are considered to create a pathway for fluid to be moved, and may be defined by a one or more components of the pump portion.

Any of the conduits herein can be deformable to some extent. For example, conduit 1112 includes elongate member 1120 that can be made of one or more materials that allow the central region 1113 of conduit to deform to some extent radially inward (towards LA) in response to, for example and when in use, forces from valve tissue (e.g., leaflets) or a replacement valve as pump portion 1104 is deployed towards the configuration shown in FIG. 2. The conduit may be stretched tightly between the expandable members in some embodiments. The conduit may alternatively be designed with a looseness that causes a greater degree of compliance. This can be desirable when the pump portion is disposed across fragile structures such as an aortic valve, which may allow the valve to compress the conduit in a way that minimizes point stresses in the valve. In some embodiments, the conduit may include a membrane attached to the proximal and distal expandable members. Exemplary materials that can be used for any conduits herein include, without limitations, polyurethane rubber, silicone rubber, acrylic rubber, expanded polytetrafluoroethylene, polyethylene, polyethylene terephthalate, including any combination thereof.

Any of the conduits herein can have a thickness of, for example, 0.5-20 thousandths of an inch (thou), such as 1-15 thou, or 1.5 to 15 thou, 1.5 to 10 thou, or 2 to 10 thou.

Any of the conduits herein, or at least a portion of the conduit, can be impermeable to blood. In FIG. 2, pump portion 1104 includes a lumen that extends from distal end 1126 of conduit 1112 and extends to proximal end 1128 of conduit 1112. The lumen is defined by conduit 1112 in central region 1113, but can be thought of being defined by both the conduit and portions of the expandable members in regions axially adjacent to central region 1113. In this embodiment, however, it is the conduit material that causes the lumen to exist and prevents blood from passing through the conduit.

Any of the conduits herein that are secured to one or more expandable members can be, unless indicated to the contrary, secured so that the conduit is disposed radially outside of one or more expandable members, radially inside of one or more expandable members, or both, and the expandable member can be impregnated with the conduit material.

The proximal and distal expandable members help maintain the conduit in an open configuration by providing radial support for the conduit, while each also creates a working environment for an impeller, described below. Each of the expandable members, when in the deployed configuration, is maintained in a spaced relationship relative to a respective impeller, which allows the impeller to rotate within the expandable member without contacting the expandable member. Pump portion 1104 includes first impeller 1116 and second impeller 1118, with first impeller 1116 disposed radially within first expandable member 1108 and second impeller 1118 disposed radially within second expandable member 1110. In this embodiment, the two impellers even though they are distinct and separate impellers, are in operable communication with a common drive mechanism (e.g., drive cable 1117), such that when the drive mechanism is activated the two impellers rotate together. In this deployed configuration, impellers 1116 and 1118 are axially spaced apart along longitudinal axis LA, just as are the expandable members 1108 and 1110 are axially spaced apart.

Impellers 1116 and 1118 are also axially within the ends of expandable members 1108 and 1110, respectively (in addition to being radially within expandable members 1108 and 1110). The impellers herein can be considered to be axially within an expandable member even if the expandable member includes struts extending from a central region of the expandable member towards a longitudinal axis of the pump portion (e.g., tapering struts in a side view). In FIG. 2, second expandable member 1110 extends from first end 1124 (proximal end) to second end 1125 (distal end).

In FIG. 2, a distal portion of impeller 1118 extends distally beyond distal end 1126 of conduit 1112, and a proximal portion of impeller 1116 extends proximally beyond proximal end 1128 of conduit 1112. In this FIG., portions of each impeller are axially within the conduit in this deployed configuration.

In the exemplary embodiment shown in FIG. 2, impellers 1116 and 1118 are in operable communication with a common drive mechanism 1117, and in this embodiment, the impellers are each coupled to drive mechanism 1117, which extends through shaft 1119 and pump portion 1104. Drive mechanism 1117 can be, for example, an elongate drive cable, which when rotated causes the impellers to rotate. In this example, as shown, drive mechanism 1117 extends to and is axially fixed relative to distal tip 1114, although it is adapted to rotate relative to distal tip 1114 when actuated. Thus, in this embodiment, the impellers and drive mechanism 1117 rotate together when the drive mechanism is rotated. Any number of known mechanisms can be used to rotate drive mechanism, such as with a motor (e.g., an external motor).

The expandable members and the conduit are not in rotational operable communication with the impellers and the drive mechanism. In this embodiment, proximal end 1121 of proximal expandable member 1108 is coupled to shaft 1119, which may be a shaft of elongate portion 1106 (e.g., an outer catheter shaft). Distal end 1122 of proximal expandable member 1108 is coupled to central tubular member 1133, through which drive mechanism 1117 extends. Central tubular member 1133 extends distally from proximal expandable member 1108 within conduit 1112 and is also coupled to proximal end 1124 of distal expandable member 1110. Drive mechanism 1117 thus rotates within and relative to central tubular member 1133. Central tubular member 1133 extends axially from proximal expandable member 1108 to distal expandable member 1110. Distal end 1125 of distal expandable member 1110 is coupled to distal tip 1114, as shown. Drive mechanism 1117 is adapted to rotate relative to tip 1114, but is axially fixed relative to tip 1114.

Pump portion 1104 is adapted and configured to be collapsed to a smaller profile than its deployed configuration (which is shown in FIG. 2). This allows it to be delivered using a lower profile delivery device (smaller French size) than would be required if none of pump portion 1104 was collapsible. Even if not specifically stated herein, any of the expandable members and impellers may be adapted and configured to be collapsible to some extent to a smaller delivery configuration.

The pump portions herein can be collapsed to a collapsed delivery configuration using conventional techniques, such as with an outer sheath that is movable relative to the pump portion (e.g., by axially moving one or both of the sheath and pump portion). For example without limitation, any of the systems, devices, or methods shown in the following references may be used to facilitate the collapse of a pump portion herein: U.S. Pat. Nos. 7,841,976, 8,052,749, the disclosures of which are incorporated by reference herein for all purposes.

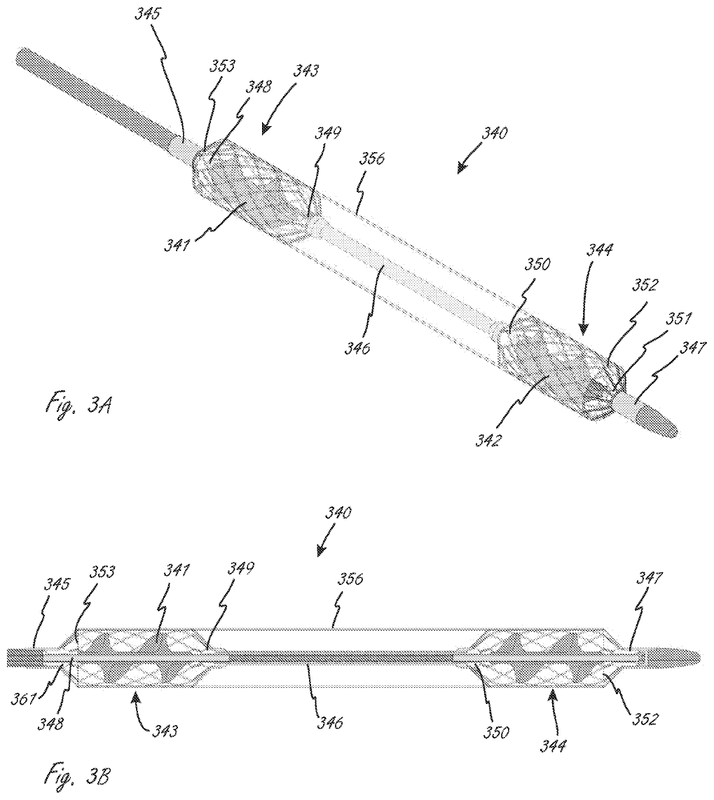

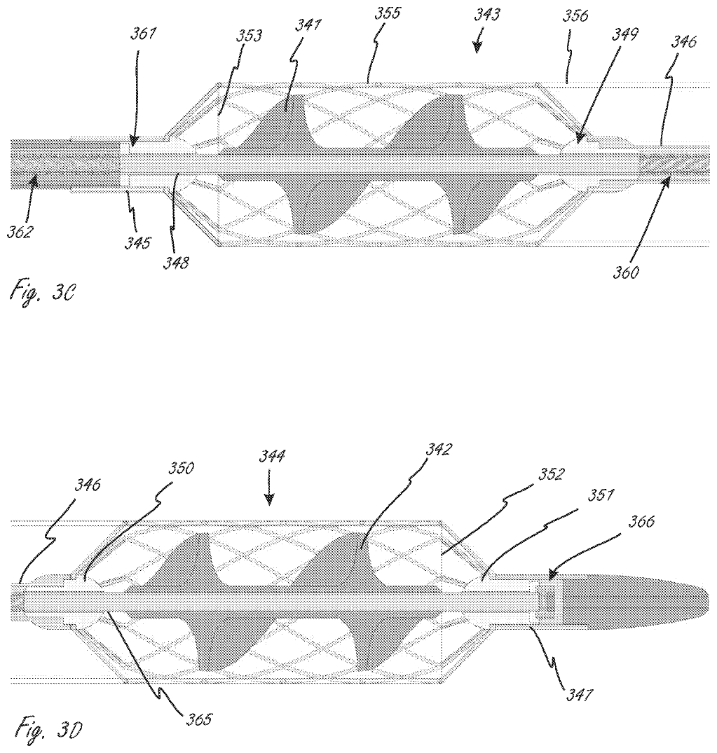

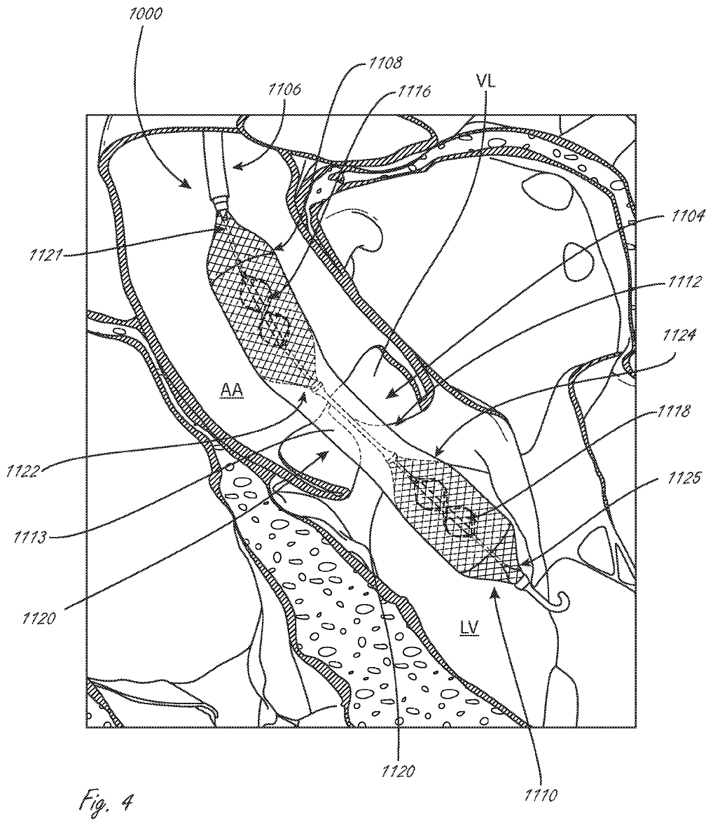

FIGS. 3A-3E show an exemplary pump portion that is similar in some ways to the pump portion shown in FIG. 2. Pump portion 340 is similar to pump portion 1104 in that in includes two expandable members axially spaced from one another when the pump portion is expanded, and a conduit extending between the two expandable members. FIG. 3A is a perspective view, FIG. 3B is a side sectional view, and FIGS. 3C and 3D are close-up side sectional views of sections of the view in FIG. 3B.

Pump portion 340 includes proximal impeller 341 and distal impeller 342, which are coupled to and in operational communication with a drive cable, which defines therein a lumen. The lumen can be sized to accommodate a guidewire, which can be used for delivery of the pump portion to the desired location. The drive cable, in this embodiment, includes first section 362 (e.g., wound material), second section 348 (e.g., tubular member) to which proximal impeller 341 is coupled, third section 360 (e.g., wound material), and fourth section 365 (e.g., tubular material) to which distal impeller 342 is coupled. The drive cable sections all have the same inner diameter, so that lumen has a constant inner diameter. The drive cable sections can be secured to each other using known attachment techniques. A distal end of fourth section 365 extends to a distal region of the pump portion, allowing the pump portion to be, for example, advanced over a guidewire for positioning the pump portion. In this embodiment the second and fourth sections can be stiffer than first and third sections. For example, second and fourth can be tubular and first and third sections can be wound material to impart less stiffness.

Pump portion 340 includes a blood flow conduit, proximal expandable member 343 and distal expandable member 344, each of which extends radially outside of one of the impellers. The expandable members have distal and proximal ends that also extend axially beyond distal and proximal ends of the impellers, which can be seen in FIGS. 3B-3D. That pumps also includes conduit 356, which has a proximal end 353 and a distal end 352. The two expandable members each include a plurality of proximal struts and a plurality of distal struts. The proximal struts in proximal expandable member 343 extend to and are secured to shaft section 345, which is coupled to bearing 361, through which the drive cable extends and is configured and sized to rotate. The distal struts of proximal expandable member 343 extend to and are secured to a proximal region (to a proximal end in this case) of central tubular member 346, which is disposed axially in between the expandable members. The proximal end of central tubular member 346 is coupled to bearing 349, as shown in FIG. 3C, through which the drive cable extends and rotates. The proximal struts of distal expandable member 344 extend to and secured to a distal region (to a distal end in this case) of central tubular member 346. Bearing 350 is also coupled to the distal region of central tubular member 346, as is shown in FIG. 3D. The drive cable extends through and rotates relative to bearing 350. Distal struts of distal expandable member extend to and are secured to shaft section 347 (see FIG. 3A), which can be considered part of the distal tip. Shaft section 347 is coupled to bearing 351 (see FIG. 3D), through which the drive cable extends and rotates relative to. The distal tip also includes bearing 366 (see FIG. 3D), which can be a thrust bearing. Working portion 340 can be similar to or the same in some aspects to working portion 1104, even if not explicitly included in the description. In this embodiment, conduit 356 extends at least as far as ends of the impeller, unlike in working portion 1104. Either embodiment can be modified so that the conduit extends to a position as set forth in the other embodiment. In some embodiments, section 360 can be a tubular section instead of wound.

In alternative embodiments, at least a portion of any of the impellers herein may extend outside of the fluid lumen. For example, only a portion of an impeller may extend beyond an end of the fluid lumen in either the proximal or distal direction. In some embodiments, a portion of an impeller that extends outside of the fluid lumen is a proximal portion of the impeller, and includes a proximal end (e.g., see the proximal impeller in FIG. 2). In some embodiments, the portion of the impeller that extends outside of the fluid lumen is a distal portion of the impeller, and includes a distal end (e.g., see the distal impeller in FIG. 2). When the disclosure herein refers to impellers that extend outside of the fluid lumen (or beyond an end), it is meant to refer to relative axial positions of the components, which can be most easily seen in side views or top views, such as in FIG. 2.

A second impeller at another end of the fluid lumen may not, however, extend beyond the fluid lumen. For example, an illustrative alternative design can include a proximal impeller that extends proximally beyond a proximal end of the fluid lumen (like the proximal impeller in FIG. 2), and the fluid lumen does not extend distally beyond a distal end of a distal impeller (like in FIG. 3B). Alternatively, a distal end of a distal impeller can extend distally beyond a distal end of the fluid lumen, but a proximal end of a proximal impeller does not extend proximally beyond a proximal end of the fluid lumen. In any of the pump portions herein, none of the impellers may extend beyond ends of the fluid lumen.

While specific exemplary locations may be shown herein, the fluid pumps may be able to be used in a variety of locations within a body. Some exemplary locations for placement include placement in the vicinity of an aortic valve or pulmonary valve, such as spanning the valve and positioned on one or both sides of the valve, and in the case of an aortic valve, optionally including a portion positioned in the ascending aorta. In some other embodiments, for example, the pumps may be, in use, positioned further downstream, such as being disposed in a descending aorta.

FIG. 4 illustrates an exemplary placement of pump portion 1104 from system 1000 from FIG. 2, and also illustrates an exemplary placement location for any of the pump portions herein. One difference shown in FIG. 4 is that the conduit extends at least as far as the ends of the impellers, like in FIGS. 3A-3D. FIG. 4 shows pump portion 1104 in a deployed configuration, positioned in place across an aortic valve. Pump portion 1104 can be delivered as shown via, for example without limitation, femoral artery access (a known access procedure). While not shown for clarity, system 1000 can also include an outer sheath or shaft in which pump portion 1104 is disposed during delivery to a location near an aortic valve. The sheath or shaft can be moved proximally (towards the ascending aorta “AA” and away from left ventricle “LV”) to allow for deployment and expansion of pump portion 1104. For example, the sheath can be withdrawn to allow for expansion of second expandable member 1110, with continued proximal movement allowing first expandable member 1108 to expand.

In this embodiment, second expandable member 1110 has been expanded and positioned in a deployed configuration such that distal end 1125 is in the left ventricle “LV,” and distal to aortic valve leaflets “VL,” as well as distal to the annulus. Proximal end 1124 has also been positioned distal to leaflets VL, but in some methods proximal end 1124 may extend slightly axially within the leaflets VL. This embodiment is an example of a method in which at least half of the second expandable member 1110 is within the left ventricle, as measured along its length (measured along the longitudinal axis). And as shown, this is also an example of a method in which the entire second expandable member 1110 is within the left ventricle. This is also an example of a method in which at least half of second impeller 1118 is positioned within the left ventricle, and also an embodiment in which the entire second impeller 1118 is positioned within the left ventricle.

Continued retraction of an outer shaft or sheath (and/or distal movement of working end 1104 relative to an outer sheath or shaft) continues to release conduit 1112, until central region 1113 is released and deployed. The expansion of expandable members 1108 and 1110 causes conduit 1112 to assume a more open configuration, as shown in FIG. 4. Thus, while in this embodiment conduit 1112 does not have the same self-expanding properties as the expandable members, the conduit will assume a deployed, more open configuration when the working end is deployed. At least a portion of central region 1113 of conduit 1112 is positioned at an aortic valve coaptation region. In FIGS. 3, there is a short length of central region 1113 that extends distally beyond the leaflets VL, but at least some portion of central region 1113 is axially within the leaflets.

Continued retraction of an outer shaft or sheath (and/or distal movement of working end 1104 relative to an outer sheath or shaft) deploys first expandable member 1108. In this embodiment, first expandable member 1108 has been expanded and positioned (as shown) in a deployed configuration such that proximal end 1121 is in the ascending aorta AA, and proximal to leaflets “VL.” Distal end 1122 has also been positioned proximal to leaflets VL, but in some methods distal end 1122 may extend slightly axially within the leaflets VL. This embodiment is an example of a method in which at least half of first expandable member 1110 is within the ascending aorta, as measured along its length (measured along the longitudinal axis). And as shown, this is also an example of a method in which the entire first expandable member 1110 is within the AA. This is also an example of a method in which at least half of first impeller 1116 is positioned within the AA, and also an embodiment in which the entire first impeller 1116 is positioned within the AA.

At any time during or after deployment of pump portion 1104, the position of the pump portion can be assessed in any way, such as under fluoroscopy. The position of the pump portion can be adjusted at any time during or after deployment. For example, after second expandable member 1110 is released but before first expandable member 1108 is released, pump portion 1104 can be moved axially (distally or proximally) to reposition the pump portion. Additionally, for example, the pump portion can be repositioned after the entire working portion has been released from a sheath to a desired final position.

It is understood that the positions of the components (relative to the anatomy) shown in FIG. 4 are considered exemplary final positions for the different components of working portion 1104, even if there was repositioning that occurred after initial deployment.

The one or more expandable members herein can be configured to be, and can be expanded in a variety of ways, such as via self-expansion, mechanical actuation (e.g., one or more axially directed forces on the expandable member, expanded with a separate balloon positioned radially within the expandable member and inflated to push radially outward on the expandable member), or a combination thereof.

Expansion as used herein refers generally to reconfiguration to a larger profile with a larger radially outermost dimension (relative to the longitudinal axis), regardless of the specific manner in which the one or more components are expanded. For example, a stent that self-expands and/or is subject to a radially outward force can “expand” as that term is used herein. A device that unfurls or unrolls can also assume a larger profile, and can be considered to expand as that term is used herein.

The impellers can similarly be adapted and configured to be, and can be expanded in a variety of ways depending on their construction. For examples, one or more impellers can, upon release from a sheath, automatically revert to or towards a different larger profile configuration due to the material(s) and/or construction of the impeller design (see, for example, U.S. Pat. No. 6,533,716, or U.S. Pat. No. 7,393,181, both of which are incorporated by reference herein for all purposes). Retraction of an outer restraint can thus, in some embodiments, allow both the expandable member and the impeller to revert naturally to a larger profile, deployed configuration without any further actuation.

As shown in the example in FIG. 4, the pump portion includes first and second impellers that are spaced on either side of an aortic valve, each disposed within a separate expandable member. This is in contrast to some designs in which a working portion includes a single elongate expandable member. Rather than a single generally tubular expandable member extending all the way across the valve, working end 1104 includes a conduit 1112 extending between expandable members 1108 and 1110. The conduit is more flexible and deformable than the pump at the locations of the impellers, which can allow for more deformation of the pump portion at the location of the leaflets than would occur if an expandable member spanned the aortic valve leaflets. Having a more flexible central region may also cause less damage to the leaflets after the pump portion has been deployed in the subject.

Additionally, forces on a central region of a single expandable member from the leaflets might translate axially to other regions of the expandable member, perhaps causing undesired deformation of the expandable member at the locations of the one or more impellers. This may cause the outer expandable member to contact the impeller, undesirably interfering with the rotation of the impeller. Designs that include separate expandable members around each impeller, particularly where each expandable member and each impeller are supported at both ends (i.e., distal and proximal), result in a high level of precision in locating the impeller relative to the expandable member. Two separate expandable members may be able to more reliably retain their deployed configurations compared with a single expandable member.

As described herein above, it may be desirable to be able to reconfigure the working portion so that it can be delivered within a 9F sheath and still obtain high enough flow rates when in use, which is not possible with some products currently in development and/or testing. For example, some products are too large to be able to reconfigured to a small enough delivery profile, while some smaller designs may not be able to achieve the desired high flow rates. An exemplary advantage of the examples in FIGS. 1, 2, 3A-3D and 4 is that, for example, the first and second impellers can work together to achieve the desired flow rates, and by having two axially spaced impellers, the overall working portion can be reconfigured to a smaller delivery profile than designs in which a single impeller is used to achieved the desired flow rates. These embodiments thus use a plurality of smaller, reconfigurable impellers that are axially spaced to achieve both the desired smaller delivery profile as well as to achieve the desired high flow rates.

Embodiments herein may be able to achieve a smaller delivery profile while maintaining sufficiently high flow rates, while creating a more deformable and flexible central region of the working portion, exemplary benefits of which are described above (e.g., interfacing with delicate valve leaflets).

Any of the blood conduits herein act to, are configured to, and are made of material(s) that create a fluid lumen therein between an first end (e.g., distal end) and a second end (e.g., proximal end). Fluid flows into the inflow region, through the fluid lumen, and then out of an outflow region. Flow into the inflow region may be labeled herein as “I,” and flow out at the outflow region may be labeled “O.” Any of the conduits herein can be impermeable. Any of the conduits herein can alternatively be semipermeable. Any of the conduits herein may also be porous, but will still define a fluid lumen therethrough. In some embodiments the conduit is a membrane, or other relatively thin layered member. Any of the conduits herein, unless indicated to the contrary, can be secured to an expandable member such that the conduit, where is it secured, can be radially inside and/or outside of the expandable member. For example, a conduit can extend radially within the expandable member so that inner surface of the conduit is radially within the expandable member where it is secured to the expandable member.

Any of the expandable member(s) herein can be constructed of a variety of materials and in a variety of ways. For example, the expandable member may have a braided construction, or it can be formed by laser machining. The material can be deformable, such as nitinol. The expandable member can be self-expanding or can be adapted to be at least partially actively expanded.

In some embodiments, the expandable member is adapted to self-expand when released from within a containing tubular member such as a delivery catheter, a guide catheter or an access sheath. In some alternative embodiments, the expandable member is adapted to expand by active expansion, such as action of a pull-rod that moves at least one of the distal end and the proximal end of the expandable member toward each other. In alternative embodiments, the deployed configuration can be influenced by the configuration of one or more expandable structures. In some embodiments, the one or more expandable members can deployed, at least in part, through the influence of blood flowing through the conduit. Any combination of the above mechanisms of expansion may be used.

The blood pumps and fluid movement devices, system and methods herein can be used and positioned in a variety of locations within a body. While specific examples may be provided herein, it is understood that that the working portions can be positioned in different regions of a body than those specifically described herein.

Although not required for the embodiments therein, there may be advantages to having a minimum axial spacing between a proximal impeller and a distal impeller. For example, a pump portion may be delivered to a target location through parts of the anatomy that have relatively tight bends, such as, for example, an aorta, and down into the aortic valve. For example, a pump portion may be delivered through a femoral artery access and to an aortic valve. It can be advantageous to have a system that is easier to bend so that it is easier to deliver the system through the bend(s) in the anatomy. Some designs where multiple impellers are quite close to each other may make the system, along the length that spans the multiple impellers, relatively stiff along that entire length that spans the multiple impellers. Spacing the impellers apart axially, and optionally providing a relatively flexible region in between the impellers, can create a part of the system that is more flexible, is easier to bend, and can be advanced through the bends more easily and more safely. An additional exemplary advantage is that the axial spacing can allow for a relatively more compliant region between the impellers, which can be positioned at, for example, the location of a valve (e.g., an aortic valve). Furthermore, there are other potential advantages and functional differences between the various embodiments herein and typical multistage pumps. A typical multistage pump includes rows of blades (sometimes referred to as impellers) in close functional spacing such that the rows of blades act together as a synchronized stage. One will appreciate that the flow may separate as it passes through the distal impeller. In various embodiments as described herein, distal and proximal impellers can be spaced sufficiently apart such that the flow separation from the distal impeller is substantially reduced (i.e., increased flow reattachment) and the localized turbulent flow is dissipated before the flow enters the proximal impeller.

In any of the embodiments or in any part of the description herein that include a distal impeller and a proximal impeller, the axial spacing between a distal end of the proximal impeller and a proximal end of the distal impeller can be from 1.5 cm to 25 cm (inclusive) along a longitudinal axis of the pump portion, or along a longitudinal axis of a housing portion that includes a fluid lumen. The distance may be measured when the pump portion, including any impellers, is in an expanded configuration. This exemplary range can provide the exemplary flexibility benefits described herein as the pump portion is delivered through curved portions of the anatomy, such as, for example, an aortic valve via an aorta.

Blood pumps, such as any of the intravascular pumps herein, may benefit from having one or more fluid paths through which fluid can flow through the device. For example without limitation, blood pumps may benefit from having one or more fluid paths through which fluid can flow to perform any of these exemplary functions: cooling rotating components (e.g., a drive cable) to prevent their overheating; flushing small particulates that may break off rotating components (e.g., a drive cable) to prevent the rotating parts from being damaged by the small particulates; lubricating rotating components (e.g., one or more bearings), and preventing blood ingress into the pump (e.g., near or at a distal end of the pump). Fluid delivery through the one or more flow paths may provide any number of these functions.

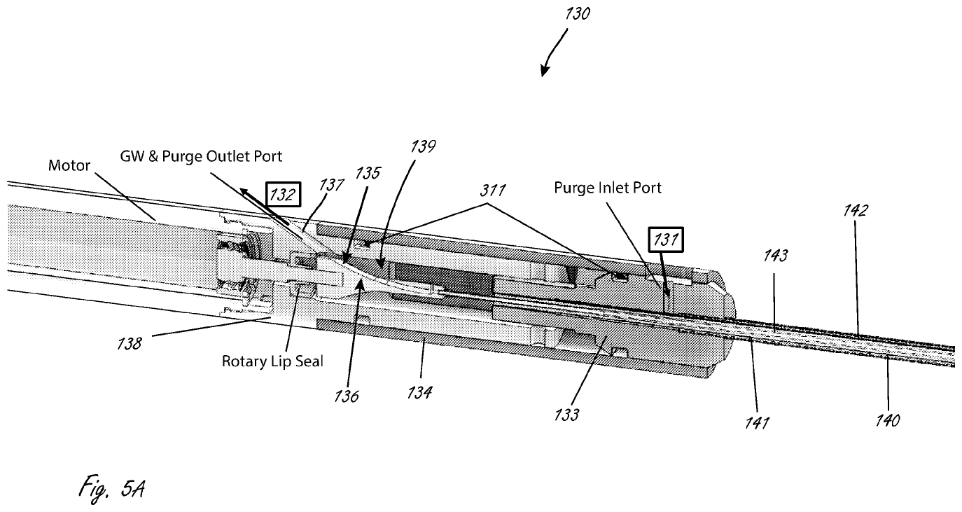

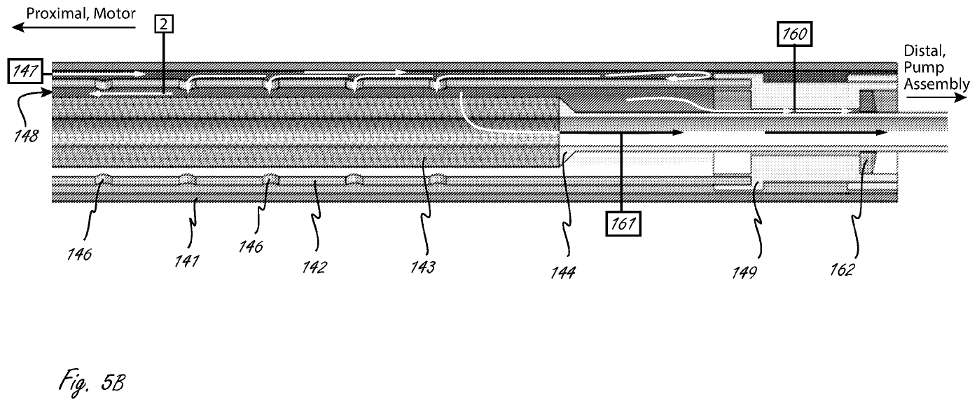

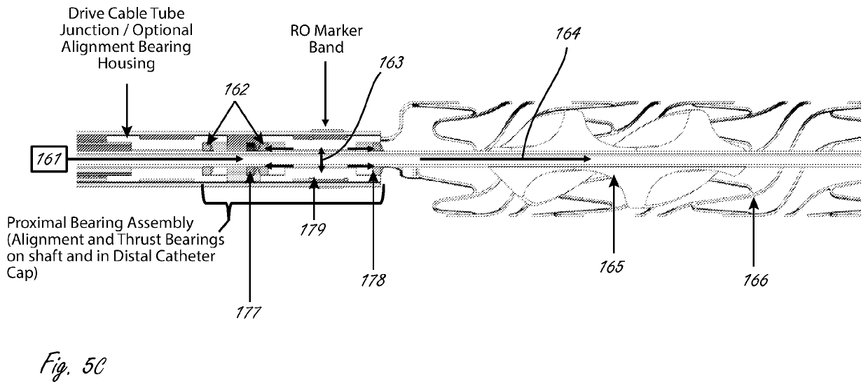

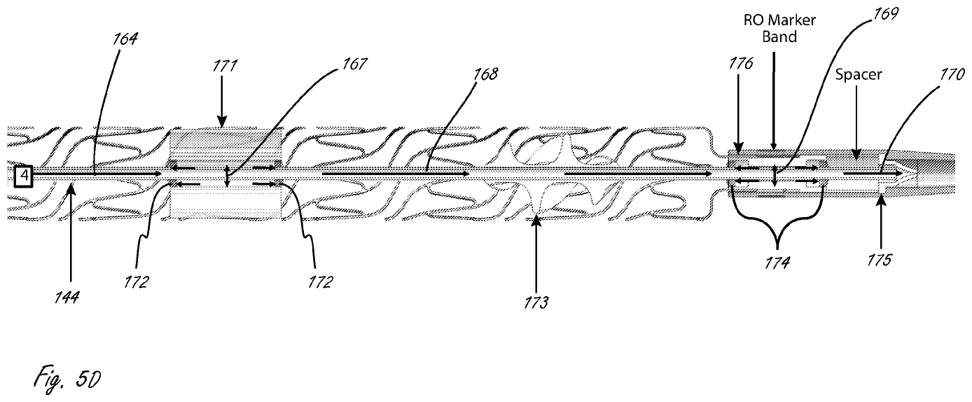

FIGS. 5A-5D illustrate an exemplary embodiment of a fluid delivery system incorporated into an exemplary fluid pump (e.g., blood pump) with a fluid inlet port and a fluid outlet port. FIG. 5A illustrates a portion of the device that is proximal to the one or more impellers, and in this embodiment includes a proximal end of a catheter, a motor assembly that causes the rotation of a drive cable and impeller(s), a fluid inlet port, and fluid outlet port, and a guidewire port that allows access to a guidewire pathway or lumen.

FIG. 5B shows a region of the device that is distal to the region shown in FIG. 5A, but includes some of the catheter components that are shown in FIG. 5A. FIG. 5C shows a region of the device distal to the region in FIG. 5B, and FIG. 5D shows a region of the device distal to the view in FIG. 5C.

While FIGS. 5A-5D illustrate different sections of an exemplary blood pumping device, it is understood that in alternative embodiments aspects of the system can vary. For example, in alternative embodiments the portion of the device with the impellers can vary and could only include a single impeller, or the expandable housing around the impeller could have a wide variety of configurations. It is understood that individual regions of the device can be incorporated by themselves into a variety of different types of blood pumps.

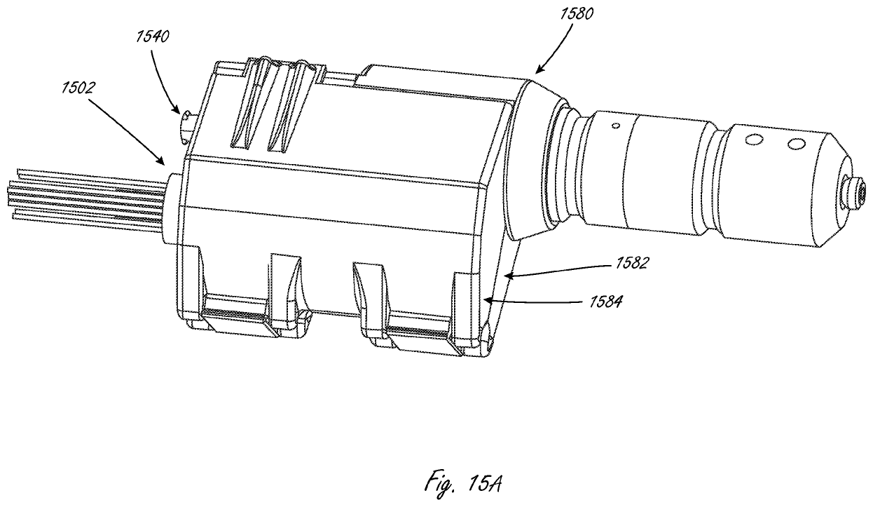

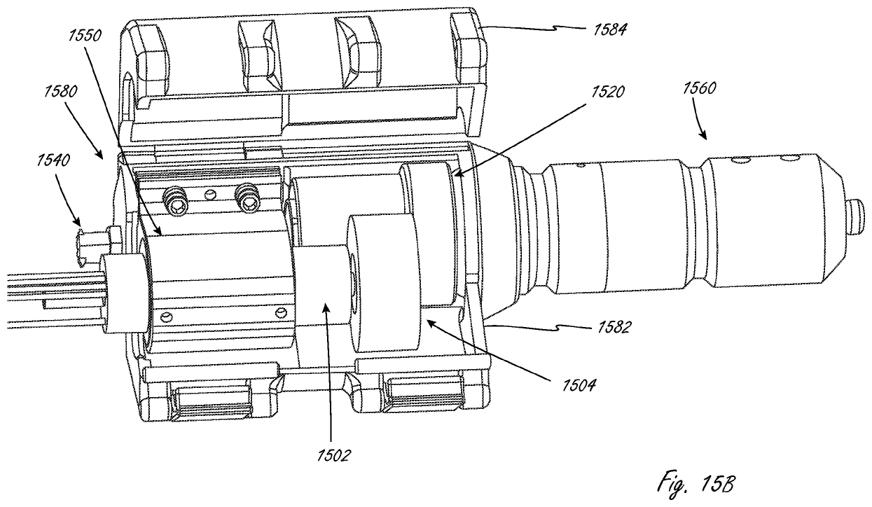

One aspect of this exemplary embodiment includes a guidewire access port that also functions as a fluid port, and in this embodiment a fluid outlet port. A motor sealing cap 138 includes, formed therein, a guidewire channel 137, including a guidewire port in a radially side surface that provides access from outside the device to channel 137. The motor sealing cap may be an optional component, and the guidewire channel 137 can alternatively be formed in a different part of the device (e.g., which may not function as a motor sealing cap). The device also includes drive cable coupler 135, which includes formed therein a guidewire channel 136, which is a portion of a guidewire pathway. Drive cable coupler 135 is rotated by the motor, and causes the rotation of drive cable 143, which causes rotation of the one or more impellers in the pump portion. These components are thus considered to be in rotational communication. Channel 137, including the guidewire port, is formed in the device and is not adapted to rotate when the motor rotates. Channel 136 formed in drive cable coupler 135 rotates when the drive cable coupler rotates. When drive cable coupler 135 is in the position shown in FIG. 15A, channel 137 is in alignment with channel 136, which allows a guidewire to be advanced through or removed from channel 137 and through channel 136. If the guidewire is being inserted, the guidewire can then be advanced further distally through the entire device and out a distal end, described in more detail below. As is also described in more detail below, the guidewire access port also acts as a fluid outlet port that allows return fluid to flow from return area 139 out of the outlet port.

One of the advantages of having the guidewire access port (part of channel 137) in the location that it is in this embodiment, is that, if needed after the pump portion has already been advanced to a location within the patient, a guidewire can be reinserted into the port and inserted all the way to and out of the distal end. Importantly, the guidewire can be reinserted without having to remove most of the device from the patient like with some rapid exchange designs, and without having to remove the motor assembly. This exemplary embodiment thus allows easy reentry of a guidewire without having to remove the motor assembly, and without having to remove the device from the subject.

Being able to reinsert the guidewire during use can be advantageous because it can, for example without limitation, allow for repositioning of the pump portion if desired or needed. For example, if the pump portion moves out of position relative to an anatomical landmark (e.g., an aortic valve), a guidewire may need to be inserted to safely reposition it relative to the anatomical landmark.

Because the guidewire path extends through a rotational component (e.g., drive cable coupler 135), it is important that the guidewire not be present in the guidewire path when the rotating component is active. The apparatuses herein can also include an automated sensing mechanism to detect the presence of the guidewire in the guidewire pathway, and/or a prevention mechanism that prevents the motor from being activated if the guidewire is in the lumen. For example without limitation, there could be a sensor that can selectively detect the presence of the guidewire in the guidewire pathway, and communicate that to a controller that prevents the motor from being activated.

In this embodiment there is a single fluid inlet channel or lumen 131 into which fluid can be delivered into the device. FIG. 5B illustrates a region of the device and illustrates different pathways the fluid can take after it has been delivered into the device. After the fluid is advanced into fluid inlet port channel 131 (which includes an inlet port), it travels through a space 147 between clean purge tube 141 and drive cable tube 142. This is considered clean input fluid. This pathway dead-ends at distal catheter cap 149. The fluid passes through the one or more apertures 146 formed in a distal region of drive cable tube 142 as shown in FIG. 5B, entering into an annular space between drive cable tube 142 and drive cable 143. Some of this fluid (optionally most of the fluid) returns in the proximal direction through this annular space, lubricating and cooling drive cable 143 and flushing potential particulate along its path. This return fluid continues to flow proximally and into area 139 shown in FIG. 5A, and continues to flow through channel 137 and out of the fluid port (which is also the guidewire access port). A fluid outlet port thus also functions as a guidewire access port in this embodiment.

While most of the fluid returns proximally to area 139, some of the fluid, after it passes through apertures 146, continues distally beyond the distal end of the drive cable 143. Some of the fluid follows proximal bearing path 160 through alignment bearing 162 to prevent blood ingress. Fluid flow along path 160 to bearing 162 can be controlled by, for example, controlling input flow pressure and throttling of the return fluid at the proximal region of the device.

Some of the fluid, after passing through apertures 146, will flow through drive cable 143, along path 161, and will continue distally through the device (e.g., through hypotube 144) and out holes to lubricate any rotating surfaces and to prevent blood ingress, described in more detail below. Guidewire lumen 145 is thus positioned to also function as a distal bearing fluid flow path.

Some fluid flows distally along path 161, as shown in FIG. 5C, and passes through holes along path 163, to lubricate one or more of bearings 162, thrust bearing 177, and alignment bearing 178. Some of the fluid continues distally in the direction of arrow 164 shown in FIG. 5C, through impeller 165 (which in this embodiment is a proximal impeller). Some of the fluid passes through apertures along path 167 to lubricate optional alignment bearings 172 that support central member 171, which may be any of the collapsible support members, including any of the central or intermediate members herein. Some fluid continues distally through the guidewire lumen in the direction of arrow 168, through optional distal impeller 173. Some fluid passes through holes along path 169 to lubricate bearings 174 that are distal to the distal impeller. Some of the fluid may also flow through valve 175 and out the distal end of the device, helping prevent blood ingress.

In this exemplary embodiment a single flow path flowing through a tubular member (path 161 that extends distally through guidewire lumen shown in FIG. 5B) leads to (is in fluid communication with) at least three distally located bearing lubricating fluid paths, 163, 167, and 169, which lubricated three axially spaced bearing regions. In some alternative embodiments, there may be a single bearing region that is lubricated, two bearing regions that are lubricated, or even more than three bearings regions that are lubricated, depending on the number of structures disposed within the expandable housing that require bearings and thus lubrication.

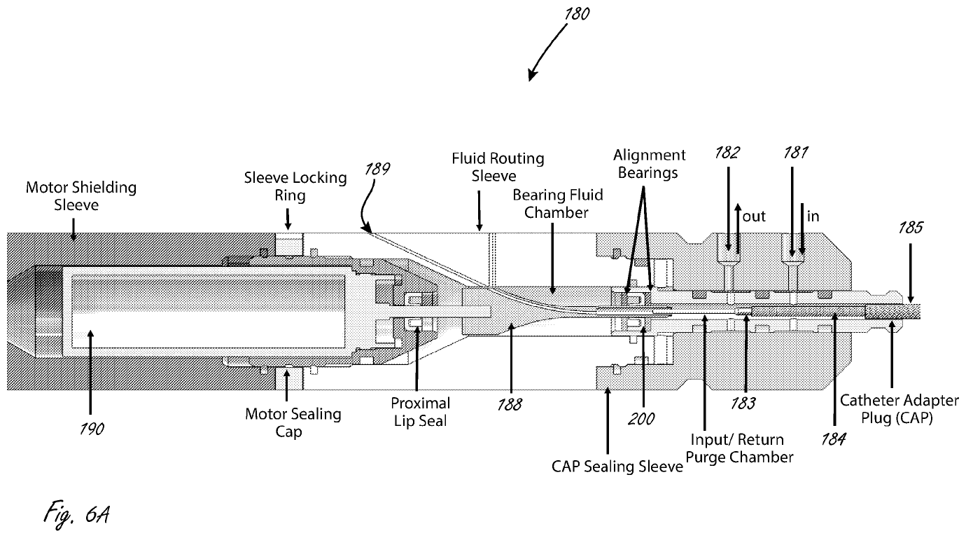

An exemplary method of using the device in FIGS. 5A-D includes inserting a guidewire near a target location (e.g., into a left ventricle via femoral artery access), then feeding the distal guidewire port over the guidewire and advancing the device over the guidewire towards the target location (e.g., an aortic valve). The method can also include removing the guidewire from the guidewire path, and coupling the proximal portion shown in FIG. 5A to a fluid inlet coupler and a fluid outlet coupler at the inlet and the outlet fluid locations, respectively. The motor can be activated to activate the one or more impellers. If the guidewire needed to be reinserted, the fluid out connector can be removed and a guidewire can be reinserted (e.g., for repositioning). The guidewire can then be removed and the fluid outlet coupler can again be put into fluid communication with the guidewire pathway. These methods or any of them individually can be incorporated into the use of any of the suitable devices herein, such as the device in FIGS. 6A and 6B. Additionally, any of the steps in any of the other exemplary methods of use herein, such as those below, may be incorporated into a use of the blood pump in this embodiment.

FIGS. 6A and 6B illustrate an exemplary embodiment of a fluid delivery system incorporated into an exemplary fluid pump (e.g., blood pump) with a first flow path with a first fluid inlet port and a first fluid outlet port. In this embodiment, however, there is also a second fluid flow path that is not in fluid communication with the first flow path. The device 180 in FIGS. 6A and 6B is similar to that shown in the embodiment in FIGS. 5A-D, except in this embodiment the fluid path 161 from FIG. 5B does not originate as fluid that flows through the drive cable. In this embodiment the fluid flow path that includes the guidewire lumen (see fluid path 196 in FIG. 6B) is in fluid communication with a separate and second fluid inlet port 189, which is also located to function as a guidewire access port, as shown in FIG. 6A. Drive cable 183 has a drive cable liner 187 on its inner surface to seal off the distal bearing flow path 196 (through the guidewire lumen). In this embodiment the guidewire access port does not function as a fluid outlet, like in FIGS. 5A-D, but as a fluid inlet port, and thus still functions as a fluid port or fluid access.

The blood pump also includes a first fluid path that includes inlet port 181 and outlet port 182 as shown in FIG. 6A. This flow path is very similar to the path in FIGS. 5A-D, except that it does not include the path through the drive cable and hypotube (i.e., does not include the guidewire lumen). The fluid is advanced through port inlet port 181, flows distally along path 197 in FIG. 6B, which is between clean purge tube 185 and drive cable tube 184. This path dead-ends at a distal catheter cap, just as in the embodiment in FIGS. 5A-D. The fluid flows through holes in drive cable tube 184, and returns proximally in the annular space between drive cable tube 184 and drive cable 183. In this part of the path the fluid lubricates and cools the drive cable and flushes potential particulate along its path, carrying them proximally to fluid exit port 182 shown in FIG. 6A. Seal 200 prevents fluid from passing proximally to seal.

Fluid flowing through the first fluid path thus lubricates and cools the drive cable, as well as flushes potential particulates and returns to exit port 182. Fluid flowing through the second fluid path travels further distally through the system, and lubricates one or more distal bearings, just as in the embodiment in FIGS. 5A-D. For example, path 199 shown in FIG. 6B is the same as path 163 in FIG. 5C, which lubricates bearings in that bearing region. While not shown, the fluid flow path distal to the view shown in FIG. 6B can be exactly the same as in FIG. 5D, thus lubricating additional bearings, and optionally exiting through a valve at a distal end of the device. This second flow path can thus also prevent ingress of blood, which is described more fully in FIGS. 5A-D.

In any of the devices herein, the pump portion can include a distal end valve distal to the impeller to seal off the distal guidewire port after the guidewire is removed, but allows for guidewire reinserting therethrough.

The following disclosure provides exemplary method steps that may be performed when using any of the blood pumps, or portions thereof, described herein. It is understood that not all of the steps need to be performed, but rather the steps are intended to be an illustrative procedure. It is also intended that, if suitable, in some instances the order of one or more steps may be different.

Before use, the blood pump can be prepared for use by priming the lumens (including any annular spaces) and pump assembly with sterile solution (e.g., heparinized saline) to remove any air bubbles from any fluid lines. The catheter, including any number of purge lines, may then be connected to a console. Alternatively, the catheter may be connected to a console and/or a separate pump that are used to prime the catheter to remove air bubbles.

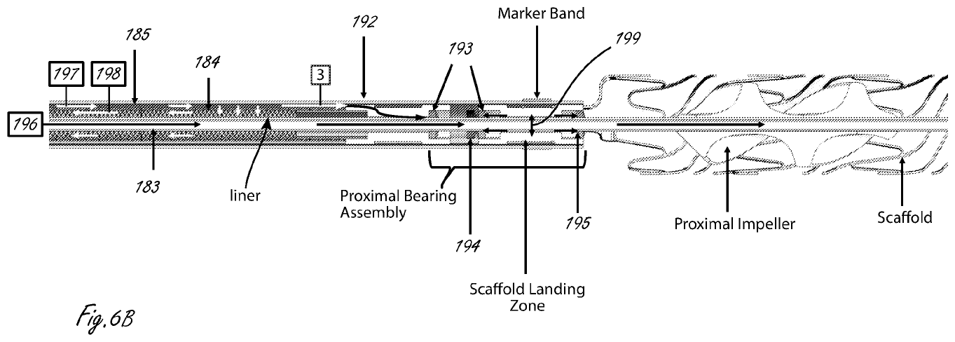

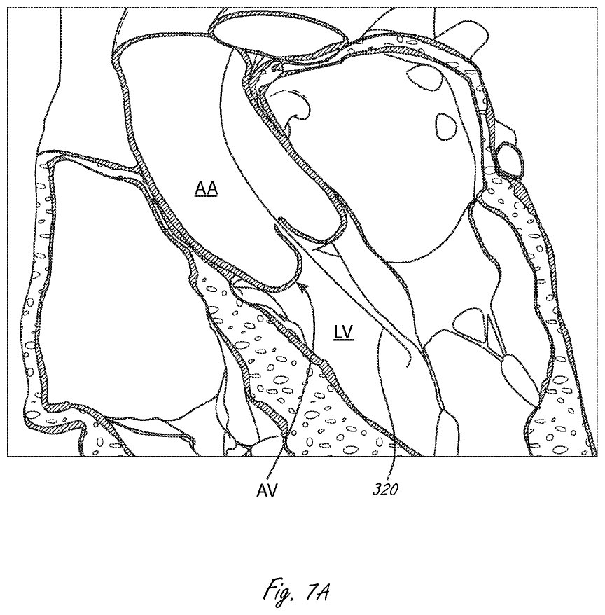

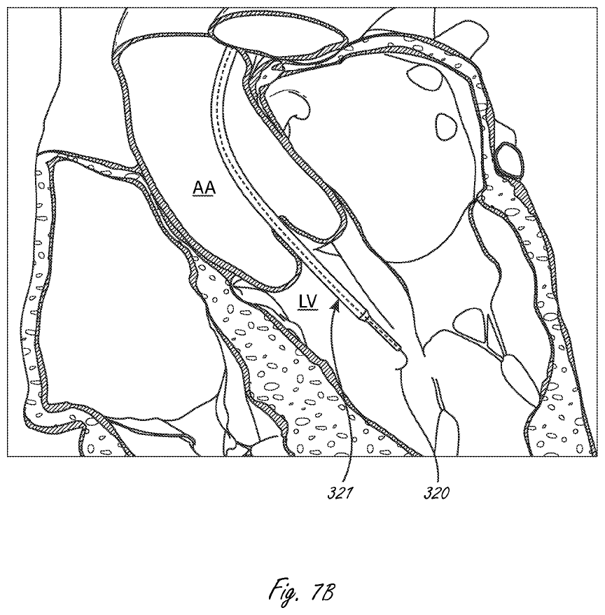

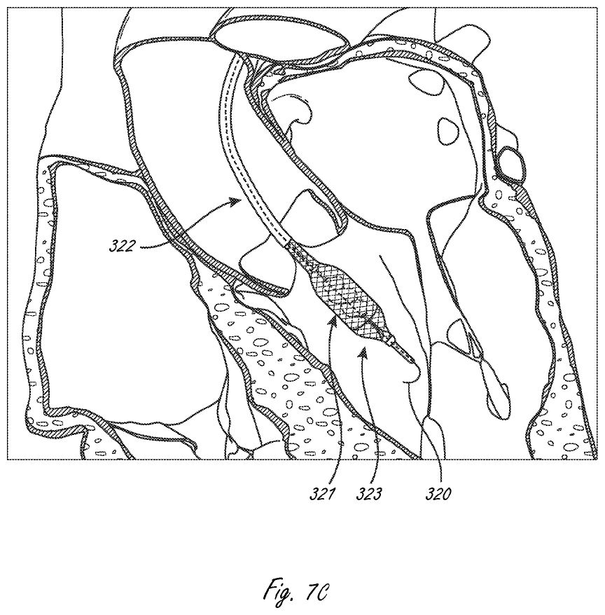

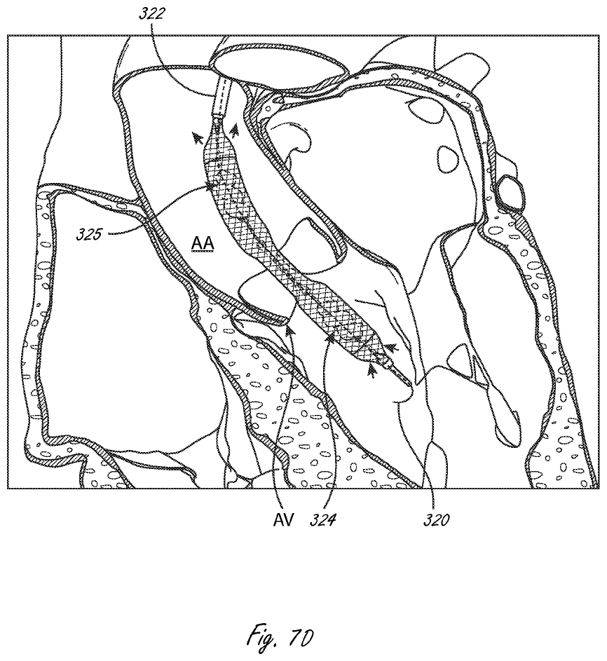

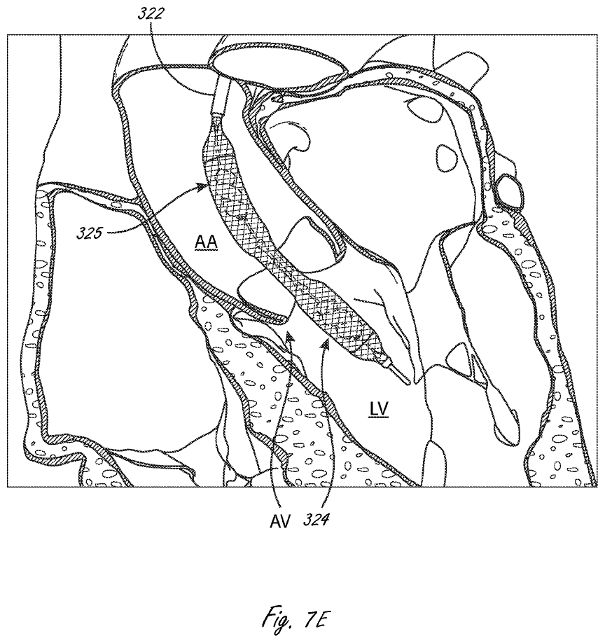

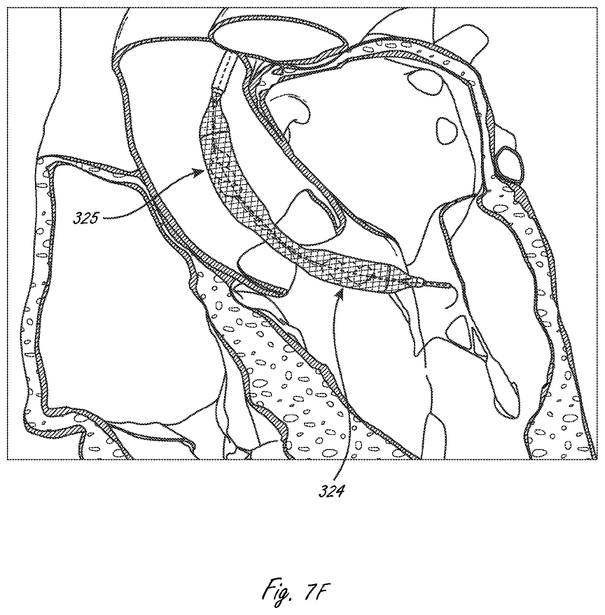

After priming the catheter, access to the patient's vasculature can be obtained (e.g., without limitation, via femoral access) using an appropriately sized introducer sheath. Using standard valve crossing techniques, a diagnostic pigtail catheter may then be advanced over a, for example, 0.035″ guide wire until the pigtail catheter is positioned securely in the target location (e.g., left ventricle). The guidewire can then be removed and a second wire 320 (e.g., a 0.018″ wire) can be inserted through the pigtail catheter. The pigtail catheter can then be removed (see FIG. 7A), and the blood pump 321 (including a catheter, catheter sheath, and pump portion within the sheath; see FIG. 7B) can be advanced over the second wire towards a target location, such as spanning an aortic valve “AV,” and into a target location (e.g., left ventricle “LV”), using, for example, one or more radiopaque markers to position the blood pump.

Once proper placement is confirmed, the catheter sheath 322 (see FIG. 7C) can be retracted, exposing first a distal region of the pump portion. In FIG. 7C a distal region of an expandable housing has been released from sheath 322 and is expanded, as is distal impeller 324. A proximal end of housing 323 and a proximal impeller are not yet released from sheath 322. Continued retraction of sheath 322 beyond the proximal end of housing 323 allows the housing 323 and proximal impeller 325 to expand (see FIG. 7D). The inflow region (shown with arrows even though the impellers are not yet rotating) and the distal impeller are in the left ventricle. The outflow (shown with arrows even though the impellers are not rotating yet) and proximal impeller are in the ascending aorta AA. The region of the outer housing in between the two impellers, which may be more flexible than the housing regions surrounding the impellers, as described in more detail herein, spans the aortic valve AV. In an exemplary operating position as shown, an inlet portion of the pump portion will be distal to the aortic valve, in the left ventricle, and an outlet of the pump portion will be proximal to the aortic valve, in the ascending aorta (“AA”).

The second wire (e.g., an 0.018″ guidewire) may then be moved prior to operation of the pump assembly (see FIG. 7E). If desired or needed, the pump portion can be deflected (active or passively) at one or more locations as described herein, as illustrated in FIG. 7F. For example, a region between two impellers can be deflected by tensioning a tensioning member that extends to a location between two impellers. The deflection may be desired or needed to accommodate the specific anatomy. As needed, the pump portion can be repositioned to achieve the intended placement, such as, for example, having a first impeller on one side of a heart valve and a second impeller on a second side of the heart valve. It is understood that in FIG. 7F, the pump portion is not in any way interfering or interacting with the mitral valve, even if it may appear that way from the figure.

Any number of purge lines may then be attached to the proximal portion of the blood pump that is disposed outside of the patient. For example, fluid inlet(s) lines and fluid outlet(s) lines may be attached to one or more fluid ports on the proximal portion of the blood pump. A purge process can then be initiated to move fluid into the blood pump through at least one fluid pathway. One or more confirmation steps can be performed to confirm the purge is operating as intended before turning on the pump. The pump assembly can then be operated, causing rotation of the one or more impellers. Any one of flow rate(s), pressure(s), and motor operation can be monitored at any time.

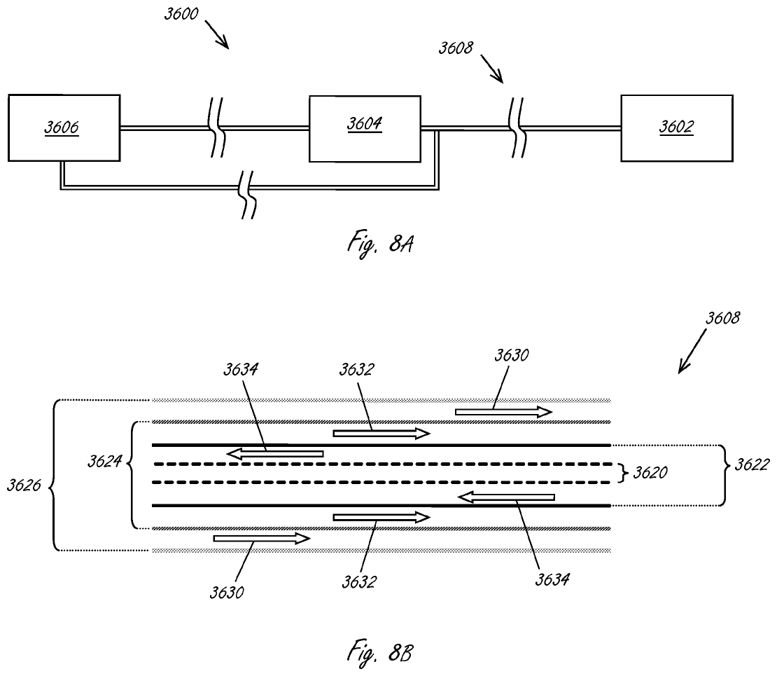

FIG. 8A schematically illustrates at least a portion of an exemplary blood pump system 3600. Any of the blood pump systems described herein may have the arrangement of system 3600. The exemplary blood pump system 3600 may include an optional external console 3606, a motor assembly 3604 and catheter portion 3608. A distal end of the catheter portion 3608 can include an intravascular blood pump 3602. The intravascular blood pump 3602 can be configured to enter a patient's vascular system to pump blood within the patient's body while the console 3606 and motor assembly 3604 remain outside of the patient's body. The console 3606 may include one or more controllers (e.g., as part of a computer) for controlling aspects of the motor assembly 3604 and blood pump 3602. The console 3606 may include a user interface (e.g., computer display) for interacting with a user. Console 3606 may be in fluid and/or electrical communication with the motor assembly 3604 via, for example, one or more electrical wires and/or one or more fluidic channels. In some examples, the console 3606 may be in fluid and/or electrical communication with the catheter portion 3608 via, for example, one or more electrical wires and/or one or more fluidic channels (e.g., bypassing the motor assembly 3604). The motor assembly 3604 may include one or more motors that are in rotational communication with one or more internal driveshafts and/or drive cables and one or more impellers of blood pump 3602. Any driveshaft and/or drive cable herein may be considered part of a rotational drive mechanism that communicates rotation from a motor to one or more impellers of the blood pump. In some cases, the motor assembly 3604 is part of a handle for controlling the blood pump 3602. In other cases, the motor assembly 3604 is a separate structural entity from a handle.

FIG. 8B schematically illustrates an exemplary sectional view of at least a portion of an exemplary catheter portion 3608, and illustrates exemplary and optional fluid pathways therein. The catheter portion 3608 may include a plurality of coaxial tubular components including a hollow driveshaft 3620, a driveshaft tube 3622, a catheter shaft 3624 and an outer sheath 3626. The hollow driveshaft 3620 can be in rotational communication with one or more impellers of a pump portion of the blood pump 3602. The hollow driveshaft 3620 can be configured to rotate by being rotatably coupled to a motor of the motor assembly 3604. The hollow driveshaft 3620 may include or define an inner lumen to accommodate, for example, a guidewire. In some cases, at least a portion of the hollow driveshaft 3620 may include a porous material through which some fluid (e.g., saline, dextrose, return fluid, etc.) may passively penetrate through the walls of the driveshaft 3620 and into the inner lumen of the driveshaft 3620. In some examples, the driveshaft 3620 walls may include a mesh material. The driveshaft tube 3622 may be sized and configured to house therein the rotatable driveshaft 3620. The catheter shaft 3624 (which may be referred to simply as a catheter, and which may include one or more layers of material) may be considered an outermost tubular member of the catheter portion along a substantially length of the catheter portion. A catheter shaft may house components of the blood pump 3602 at a distal portion of the catheter 3624, such as one or more bearing assemblies of the blood pump 3602. Once, in position, the sheath 3626 may be retracted proximally to allow expansion of the optionally expandable portions of the blood pump 3602. The motor assembly 3604 is generally adapted to rotate the driveshaft 3620 and not adapted to rotate the driveshaft tube 3622, catheter shaft 3624 and/or sheath 366.

Depending on the particular design of the catheter portion, the catheter portion may include one or more fluid pathways to facilitate fluid flow in and through one or more annular spaces between components of the catheter portion 3608. For example, clean fluid (e.g., clean saline) may flow (e.g., by being pumped with a pump) toward the blood pump 3602 via a sheath fluid pathway 3630 between the sheath 3626 and the catheter shaft 3624. Fluid flow through the sheath fluid pathway 3630 may prevent blood from stagnating and forming clots in the annular space between the sheath 3626 and the catheter shaft 3624 at a distal end of the sheath 3626. Fluid from the sheath fluid pathway 3630 may enter the patient's body with no substantial return fluid pathway. Clean purge fluid (e.g., saline pumped from a saline bag disposed outside the patient) may also flow (e.g., by being pumped) toward the blood pump 3602 via a catheter clean fluid pathway 3632 between the catheter shaft 3624 and the driveshaft tube 3622. Some or all of the fluid in the catheter clean fluid pathway 3632 may return from the blood pump 3602 via a return fluid pathway 3634 (which may be referred to in any embodiment herein as a waste fluid pathway). Flowing fluid through the catheter fluid pathway 3632 and return fluid pathway 3634 may cool and/or lubricate moving components (e.g., the rotating driveshaft 3620 and bearings) within the blood pump 3602. The catheter clean fluid pathway 3632 and return fluid pathway 3634 may flush and keep possible debris (e.g., from wear of rotating components) from entering the patient's body. In some examples, where a wall of the driveshaft 3620 has some porosity, fluid within the return fluid pathway 3634 may passively enter the inner lumen of the driveshaft 3620.

In any of the embodiments herein, a driveshaft, a driveshaft tube, a catheter shaft and optionally an outer sheath may all be co-axial.

Optionally, clean fluid for the sheath fluid pathway 3630 and the catheter fluid pathway 3632 may be provided by a console 3606, which may include one or more clean fluid sources (e.g., saline bags) and a pump assembly (e.g., peristaltic pump assembly) for moving clean fluid distally toward the blood pump 3602. In some examples, the clean fluid may be provided to the catheter portion 3608 through a catheter fluid inlet and a sheath fluid inlet between the motor assembly 3604 and the blood pump 3602. In some cases, one or both of the catheter fluid inlet and the sheath fluid inlet are part of (or connected to) the motor assembly 3604. In some examples, the return fluid pathway 3634 may flow through the motor assembly 3604 and toward a waste reservoir, which optionally may be connected to (or part of) such as by being secured to, the console 3606.

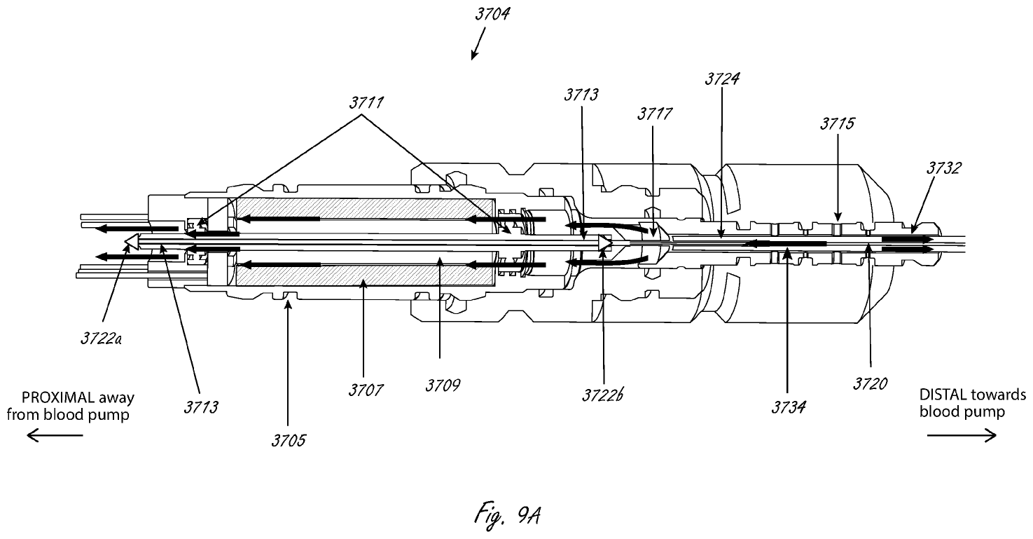

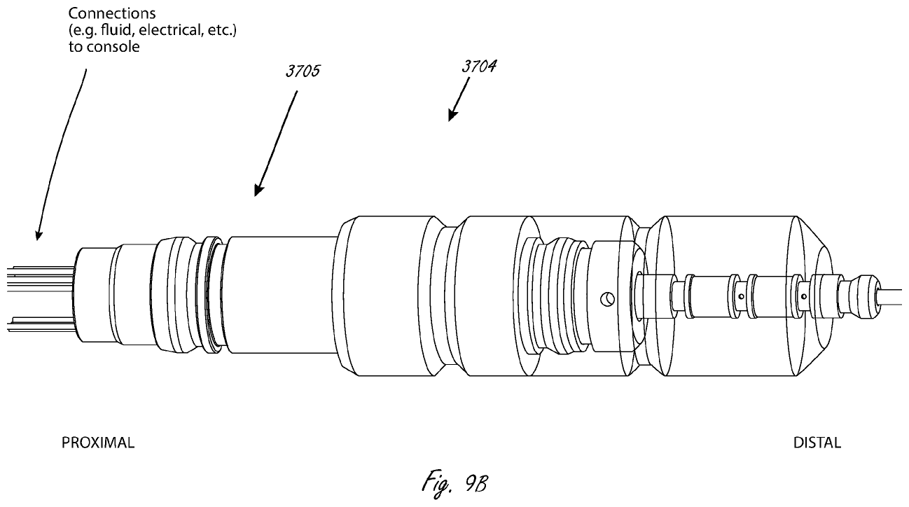

In some examples, the motor assembly 3604 may be configured to allow fluid to pass therethrough to cool, lubricate and/or flush various internal components of the motor assembly 3604, as well as optionally providing a pathway for at least some of the return fluid through the system. FIGS. 9A and 9B illustrate an exemplary motor assembly 3704 showing an exemplary fluid pathway therethrough. Clean fluid (e.g., from the console) can enter an inlet 3715 that is, in this case, at a distal portion of the motor assembly 3704. The clean purge fluid 3732 can travel in a distal direction toward the blood pump radially outside of the catheter shaft 3724 and the inner hollow driveshaft 3720. The hollow driveshaft may be rotationally coupled with one or more impellers of the blood pump. In some examples, the clean purge fluid 3732 moved toward the blood pump forms the catheter fluid pathway for cooling, lubricating and/or flushing one or more components of the blood pump, such as the driveshaft and/or one or more bearings in the pump section, such as any of the pump sections incorporated by reference herein.

At least some (e.g., nominally all) of the clean purge fluid 3732 returns from the blood pump as return purge fluid 3734 through the lumen of catheter shaft 3724. The hollow driveshaft 3720 may be at least partially permeable to fluid such that some of the fluid within the catheter shaft 3724 passively seeps into the lumen of the hollow driveshaft 3720. The return purge fluid 3734 can travel proximally through the catheter shaft 3724 and exit an intersection region 3717.

From the intersection region 3717, the return fluid can be directed in an annular space around a hollow motor shaft 3713 that is rotationally coupled to the hollow driveshaft 3720. The return purge fluid can then be directed through spaces between rotational elements (e.g., balls) of a first bearing and into an annular space between a stator 3707 and a rotor 3709 of a motor 3705. The motor 3705 can be configured to rotate the hollow motor shaft 3713, which is rotationally coupled to the hollow driveshaft 3720. Moving further proximally, the return purge fluid can exit the motor 3705 through spaces between rotational elements (e.g., balls) of a second bearing and exit a proximal end of the motor assembly 3704. Once exited the motor assembly 3704, the return fluid may be directed to a waste reservoir, for example, at an external console of the blood pump system.

In some examples, the motor assembly 3704 optionally includes one or more one-way valves (e.g., 3722a and 3722b), which can prevent fluid from entering the hollow motor shaft 3713 within the motor 3705. This may keep that lumen of the hollow motor shaft 3713 clean in the event a guide wire needs to be advanced distally back through the blood pump through this lumen.

An exemplary benefit of the configuration shown in FIGS. 9A and 9B is that the return purge fluid 3734 running through the motor 3705 can keep the motor bearings 3711 lubricated. The return purge fluid 3734 may flush out debris in the bearings 3711. In some examples, rotary seals are not necessary since the motor 3705 can have sealed electronics and can function normally while filled with purge fluid. In this example, the return fluid path through the motor 3705 may provide some degree of motor cooling, although creating a pathway for return fluid to flow out of the blood pump system may be a more important design feature.

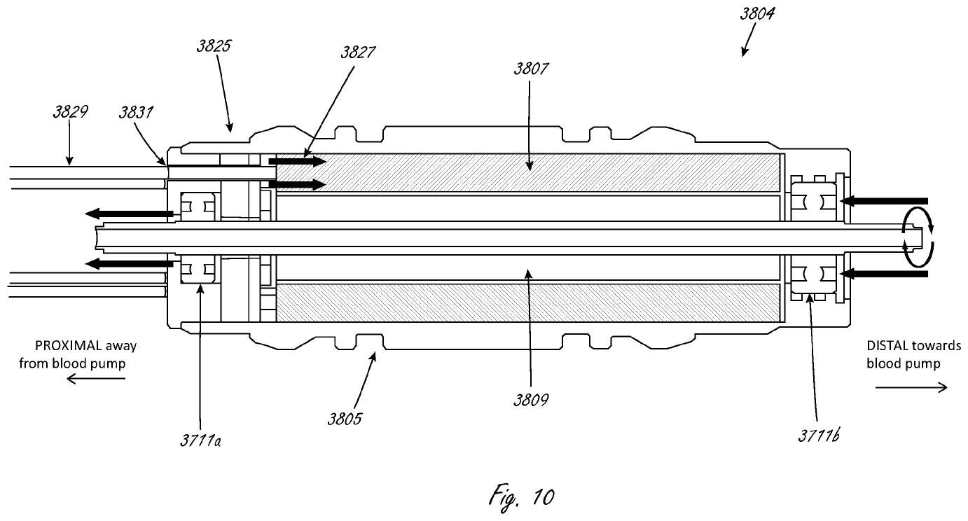

FIG. 10 shows an exemplary motor assembly 3804 of an intravascular blood pump, illustrating a possible fluid ingress site to a stator 3807. In this example, the fluid may be able to enter the stator 3807 around a wire connection region 3825 of the stator 3807 where wires 3829 exit the stator 3807. For example, fluid may enter spaces between the wires 3829 and the housing surrounding the motor 3805 if potting 3831 does not provide a sufficient fluid impermeable seal. Another possible path of fluid ingress may be at junctions between parts of the housing surrounding the motor 3805, such as the exemplary circumferential weld junction 3833. If such weld regions are improperly formed, these regions may also allow fluid to enter the stator 3807. Fluid ingress into the stator 3807 may cause an electrical short and/or cause the stator 3807 to corrode.

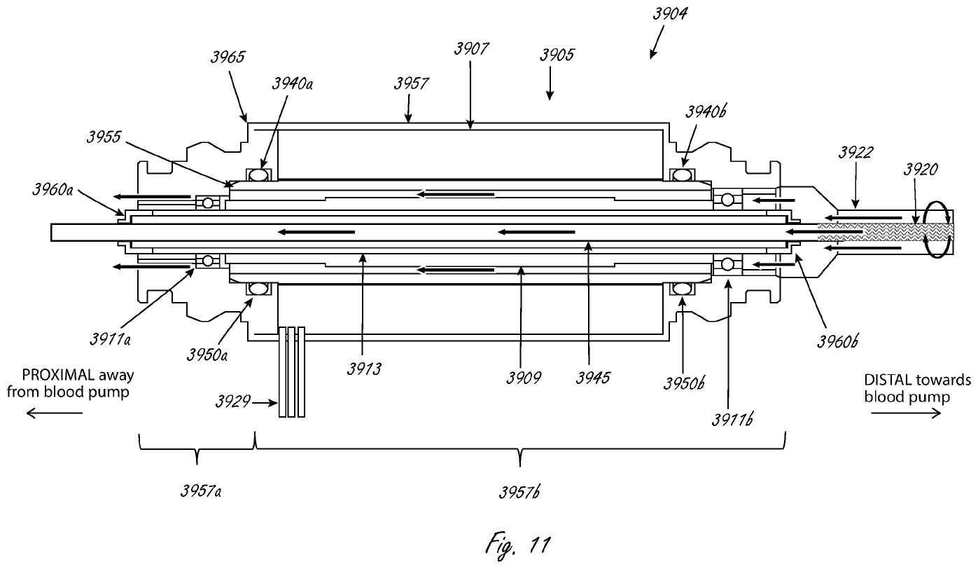

FIG. 11 illustrates an exemplary motor assembly 3904 configured to fluidically isolate the stator from the fluidic pathway, thereby addressing the fluid ingress problems described with respect to FIG. 10. In this example, a fluid impermeable layer 3955 is positioned between the stator 3907 and the rotor 3909. The fluid impermeable layer 3955 may act as a barrier to prevent fluid from the fluid pathway within the motor from reaching the stator 3907. The fluid impermeable layer 3955 may have a cylindrical shape with the rotor 3909 positioned within the inner lumen of the fluid impermeable layer 3955. The fluid impermeable layer 3955 may be made of a non-electrically conductive material to prevent interference with the functioning of the motor 3905. In some cases, the fluid impermeable layer 3955 comprises a polymer material. As shown, fluid (e.g., return purge fluid) may flow radially within the impermeable layer 3955 and radially outside of the rotor 3909.

The fluid impermeable layer 3955 may be secured in place within the motor assembly 3904. For example, the housing 3957 of the motor assembly 3904 may include multiple sections that are coupled (e.g., bonded, welded, or otherwise coupled) together with the fluid impermeable layer 3955 disposed therein. In the non-limiting example shown, the housing 3957 includes a first housing portion 3957a and a second housing portion 3957b that are coupled together by a circumferential weld 3965, thereby encasing the fluid impermeable layer 3955 within the housing 3957. In alternative examples, the housing may include other arrangements of a plurality of housing portions that are coupled together (at one or more coupling locations) to secure the fluid impermeable layer therein. For example, the housing may include more than two housing portions coupled together. Additionally, for example, first housing portion 3975a and second housing portion 3975b may be coupled (e.g., welded) in a distal portion of the motor assembly.

The motor assembly 3904 may include one or more sealing elements or members (e.g., one or more O-rings) strategically placed to prevent fluid from reaching the stator 3907. The exemplary motor assembly 3904 includes a first sealing element (e.g., O-ring in this example) 3950a proximally located with respect to the stator 3907 and a second O-ring 3950b distally located with respect to the stator 3907. These O-rings 3950a and 3950b positioned either side of the stator 3907 can be sized and positioned to prevent fluid from reaching the stator 3907 from axial directions. The O-rings 3950a and 3950b may be positioned within annular groves 3940a and 3940b of the motor assembly housing 3957. In some examples, the O-rings 3950a and 3950b are in contact with and form a seal with the fluid impermeable layer 3955.

In some examples, wires 3929 for the stator 3907 may be configured to extend radially outward from the stator 3907 so that their entry points into the stator 3907, which may be easy entry points for fluid ingress, are situated away from the fluid path. The wires 3929 may extend through a slot on an outer portion of the motor assembly housing 3957. In some examples, the wires 3929 enter a sealed handle compartment.

Example fluid pathways of return fluid from the blood pump through the exemplary motor assembly 3904 are shown in arrows in FIG. 11. As shown, the return fluid can enter from a distal side of the motor 3905 (relative to the console) via driveshaft tube 3922 that surrounds the rotatable driveshaft 3920. The driveshaft 3920 may be permeable to fluid such that some fluid within the driveshaft tube 3922 may passively enter within the inner lumen of the driveshaft 3920. The return fluid from the driveshaft tube 3922 and driveshaft 3920 travel two different fluid pathways through the motor 3905. In a first fluid pathway, fluid can travel though a distal bearing 3911b (e.g., to cool, lubricate and/or flush the distal bearing 3911b), in an annular space between the fluid impermeable layer 3955 and the rotor 3929 (e.g., to cool, lubricate and/or flush the rotor 3909 and/or stator 3907), through a proximal bearing 3911a (e.g., to cool, lubricate and/or flush the proximal bearing 3911a), and out a proximal end of the motor assembly 3904.

In a second fluid pathway through the motor 3905, fluid may travel through a hypotube 3945, which is rotatably coupled to the driveshaft 3920 (as shown), and out the proximal end of the motor assembly 3904. The hypotube 3945 can be positioned within the lumen of a hollow motor shaft 3913, which is rotatably coupled to the rotor 3909. The hypotube 3945 may be impermeable to fluid passage therethrough, thereby preventing fluid from entering in the annular space between the hypotube 3945 and the hollow motor shaft 3913. The hypotube 3945 may be rotatably coupled to the hollow motor shaft 3913 by couplers 3960a and 3960b, which may have annular shapes sized to accommodate the hypotube 3945 positioned therethrough. The hypotube 3945 may be configured to accommodate a guidewire therethrough. Return fluid exiting from the proximal side of the motor assembly 3904 can travel to a waste fluid line toward a waste fluid reservoir, for example.

Purge fluid as used herein may also be referred to as a lubricating fluid, flushing fluid and/or a cooling fluid, and vice versa.

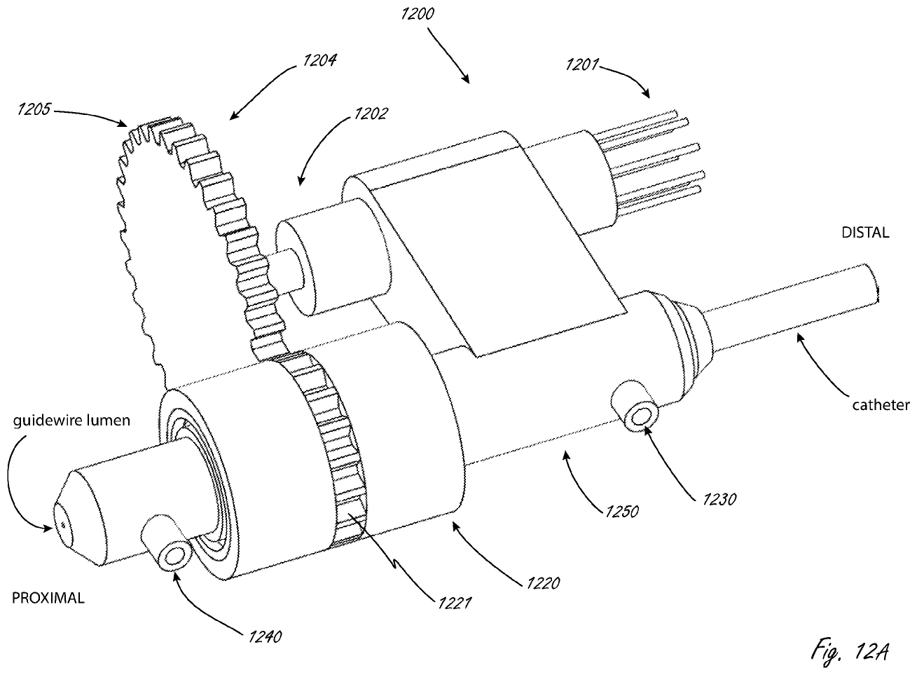

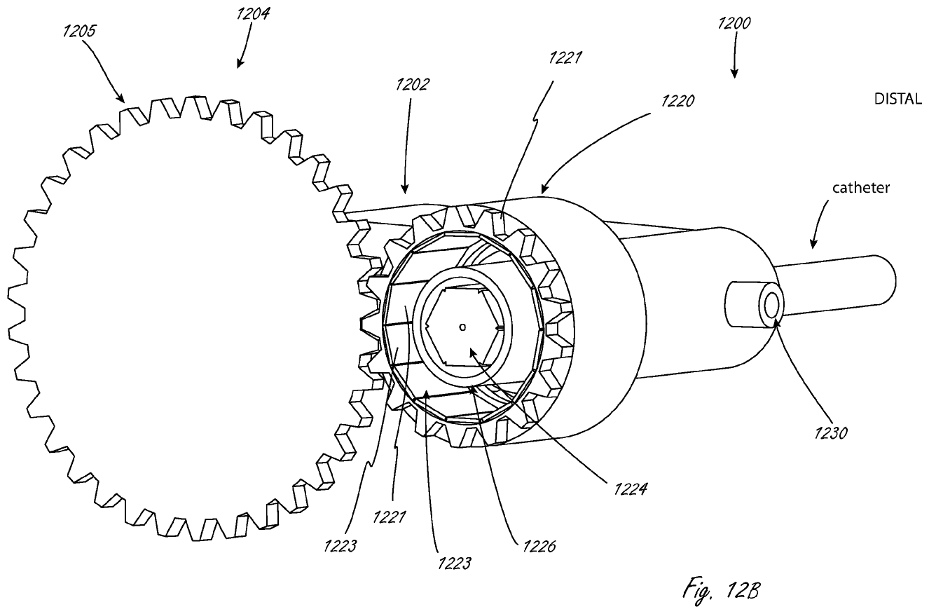

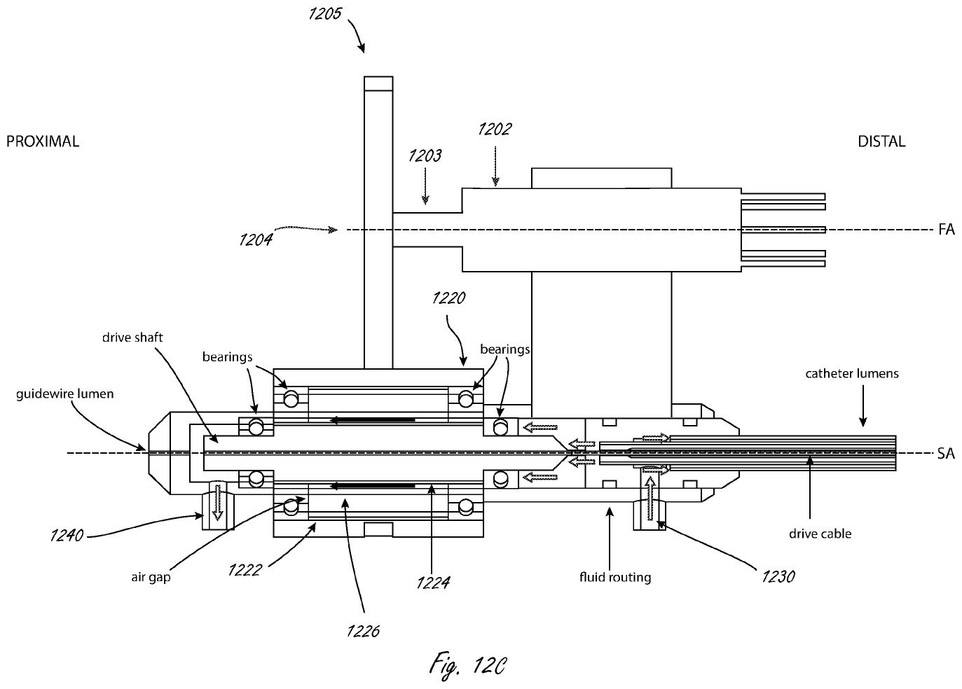

FIGS. 12-14 illustrate a portion of an exemplary intravascular blood pump in which a motor is isolated from a return fluid pathway. In some instances, return fluid and any particulate that may be in the return fluid may damage a motor or prevent the motor from functioning properly or optimally. By electrically isolating the motor from the return fluid, damage or malfunctioning due to exposure to the return fluid and any particulates therein can be prevented. The motor in this example may include a stator and a rotor, as is common with electric motors, and may be separately cooled with a fluid other than a catheter return fluid.

Blood pump system 1200 includes external motor 1202 that includes a stator and rotor therein, with electrical wires 1201 coupled to motor 1202. The motor rotor is coupled to motor shaft 1203 and causes the rotation thereof. Motor shaft 1203 is coupled to mechanical driving member 1204, which in this example is a geared member that includes teeth 1205. Motor 1202, motor shaft 1204 and mechanical driving member 1204 are adapted to rotate around first axis FA, as shown in FIG. 12C. The portion of the blood pump system 1200 shown in FIG. 12A is generally configured to stay outside of the patient during use, although concepts herein may be applied to or useful for on-board or internal motor assemblies that are disposed inside a patient.

Mechanical driving member 1204 is mechanically coupled with mechanical driven member 1220, which in this example is also geared and includes teeth 1221, such that rotation of driving member 1204 causes the rotation of driven member 1220. In this example the mechanical coupling includes geared members with teeth making direct contact, but in other examples the mechanical coupling may be or include other mechanically coupling features that are directly or indirectly mechanically coupled. When rotated, mechanical driven member 1220 rotates about second axis SA, as shown in FIG. 12C. Mechanical driven member 1220 includes teeth in a region that is in between distal and proximal ends of the driven member 1220. In this example, driven member 1220 has a cylindrical outer profile in regions that are distal to and proximal to the teeth, as shown in FIG. 12A. In this example, driven member 1220 has gears that do not extend all of the way from a proximal end to a distal end of the driven member.

It may beneficial that the blood pump systems herein have a gear ratio (for either gear or friction drives) that helps minimize the motor rotational speed to, for example, help reduce motor vibration or reduce wear. The sizes of gears or rollers with respect to one another determines the torque or speed multiplication, which effects motor performance. For example, the relative outer dimensions of the driven and driving members can be selected or chosen to provide a desired speed multiplication between the driven and driving members, such as greater than 1:1, such as about 1:1.5, or about 2:1. In the examples herein, the driving member has a greater outer dimension (e.g., diameter) than the driven member, which allows the driving member (which is coupled to the motor) to rotate slower than the driven member.

As shown in FIG. 12B, mechanical driven member 1220 is coupled to rotatable outer cylindrical magnet assembly 1222, with the outer magnet assembly 1222 coupled to and optionally connected to an inner surface of mechanical driven member 1220. Outer magnet assembly 1222 includes a plurality of magnetic members 1221 and 1223 with, in this example, alternating polarity around the entirety of outer magnet assembly 1222, as shown. Rotation of mechanical driven member 1220 causes rotation of outer magnet assembly 1222 about second axis SA. This rotation may be 1:1, or in some embodiments it (as well as the inner magnetically assembly) may be magnetically geared to multiply the torque or the speed like a gearbox.

Blood pump system 1200 also includes a rotatable drive shaft 1224 or other rotatable component rotationally coupled to a drive shaft, which in this example includes a plurality of magnetic members secured to an outer surface of the drive shaft with alternating polarity around the shaft, as shown in FIG. 12B. The alternating polarity of the magnetic members coupled to or part of the drive shaft, which magnetically responds when the outer magnet assembly is rotated, causes the drive shaft to rotate when the outer magnetic assembly 1222 rotates (which also causes the rotation of the one or more impellers in the distal pump of the blood pump 1200). The distal pump may be any of the pump portions herein (which may also be referred to herein as blood pumps), such as an expandable blood pump with one or more expandable impellers. The inner and outer magnetic assemblies are magnetically coupled and an axial spacing or gap exists between the inner and outer magnetic assemblies.

The magnetic assemblies herein may alternatively include a variety of different magnetic member arrangements than those described herein and still provide the magnetic coupling functionality between a first and a second magnetic assemblies that is provided herein.

In alternative examples, the magnetic assemblies need not be outer and inner, but rather may be axially face-to-face, with the magnetic members rotated 90 degrees relative to the orientation in the example in FIGS. 12A-12C. In these alternatives, the magnetic assemblies may be referred to as distal and proximal magnetic assemblies. For example, a mechanical driven member may be coupled to a proximal magnetic assembly, the rotation of which may cause a distal magnetic assembly to rotate. The distal magnetic assembly is rotationally coupled to a drive shaft in these alternatives. The return fluid can thus still be isolated from the motor in these alternatives. All other aspects of any of the blood pumps systems herein can be incorporated into these alternative designs.

The external portion of blood pump system 1200 also includes a stationary (non-rotating) return fluid member 1226 disposed about the inner magnetic assembly and drive shaft 1224. Return fluid member 1226 acts as a fluid barrier between the inner and outer magnetic assemblies, and is part of the return fluid pathway through which return fluid flows. Return fluid member 1226, and other outer stationary return fluid member herein, may be polymeric to avoid the magnetic flux from inducing heating in the return fluid member.

Additionally, as shown, the entire assembly comprises a guidewire lumen therethrough to allow for co-axial guidewire access, the innermost lumen of which can be seen in FIG. 12B.

The exemplary blood pump system 1200 includes a fluid inlet 1230 into which clean purge fluid may be delivered, exemplary details of which are included herein. Clean purge fluid may be moved distally between an outer catheter shaft and an inner drive cable tube in a catheter fluid pathway, an example of which is shown in FIG. 8B. Return fluid may flow along a return fluid pathway, which may include a volume between a drive shaft tube and a driveshaft, an example of which is shown in FIG. 8B. As shown in FIG. 12C, return fluid may then flow around drive shaft 1224, through ball bearings or bushings, and between return fluid member 1226 and drive shaft 1224, as shown by the arrows in FIG. 12C. After flowing through another set of ball bearings or bushings, the return fluid exits at return fluid outlet 1240, which in this example is a lateral outlet but in alternatives it may be extending proximally from a proximal end of the external assembly.

In this example, purge fluid inlet 1230 is distal to the mechanical driven member 1220 and return fluid outlet 1240 is proximal to the mechanical driven member 1220.

In this and other examples herein, the drive shaft (e.g., drive shaft 1224) is rotationally coupled with a drive cable (in this example indirectly), as is shown in FIG. 12C.

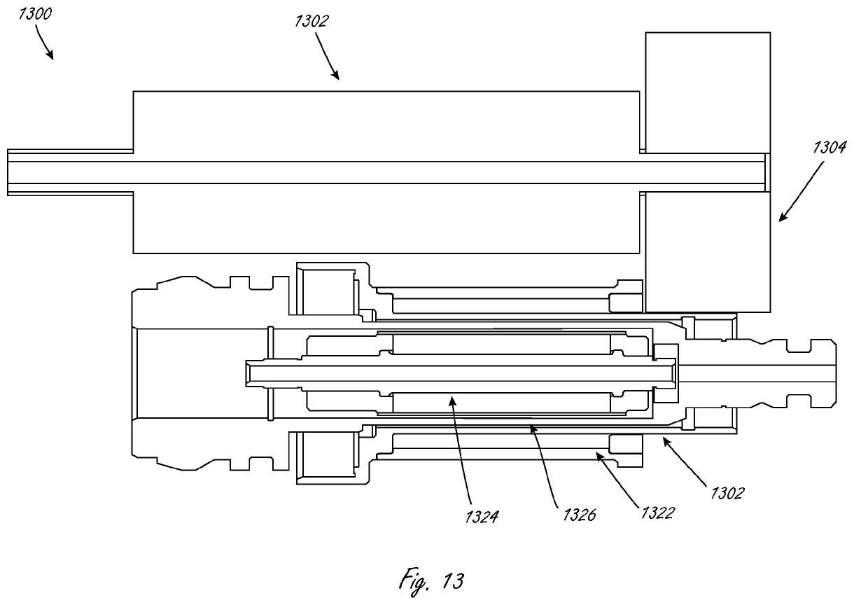

FIG. 13 is a sectional view of a portion of an exemplary motor assembly of a blood pump system. While not all components are shown for clarity, any other suitable feature or component herein may be incorporated into the blood pump system 1300. Blood pump system 1300 includes motor body 1302 that includes a stator and a rotor, wherein the rotor is coupled to the motor shaft, and the motor shaft is coupled to mechanical driving member 1304, similar to the example in FIGS. 12A-12C. Rotation of mechanical driving member 1304 causes rotation of mechanical driven member 1302, to which a first magnetic assembly 1322 (which includes a plurality of magnetic members of alternating polarity around the assembly) is coupled. In this example, first magnetic assembly 1322 (which in this example is an outer cylindrical magnetic assembly, but may be a proximal or distal magnetic assembly) is coupled to an outer surface of the mechanical driven member 1302, as shown in FIG. 13. First magnetic assembly 1322 is magnetically coupled to a second magnetic assembly (which in this example is an inner cylindrical magnet assembly), the rotation of which causes rotation of drive shaft 1324, similar to the example in FIGS. 12A-12C. Alternatively, shaft 1324 may be a rotatable component that is rotationally coupled to a drive shaft. As can be seen, the driving member in this example has a larger diameter than the driven member diameter, which provide the speed multiplication described herein. In some examples the speed multiplication is between 1:1 and 3:1, such as about 2:1. In this example, as shown, the driving member interfaces with a reduced diameter portion of the outer magnetic assembly, which allows the roller driving member to have the desired diameter and couple the driven member to a first magnetic coupling assembly.

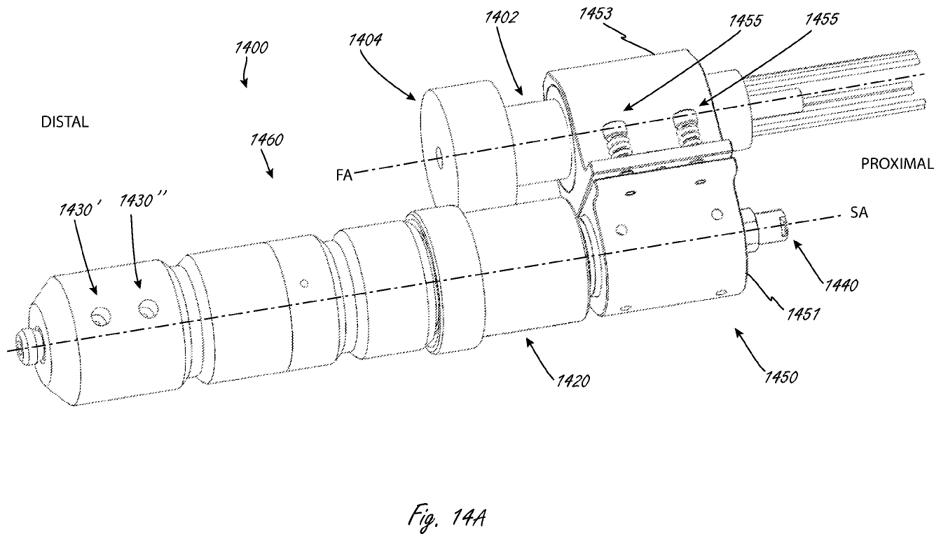

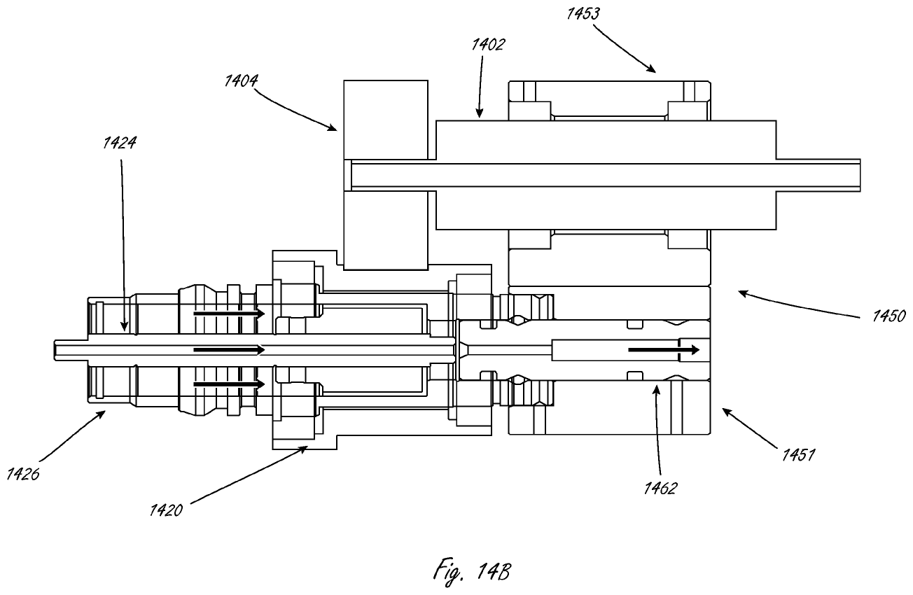

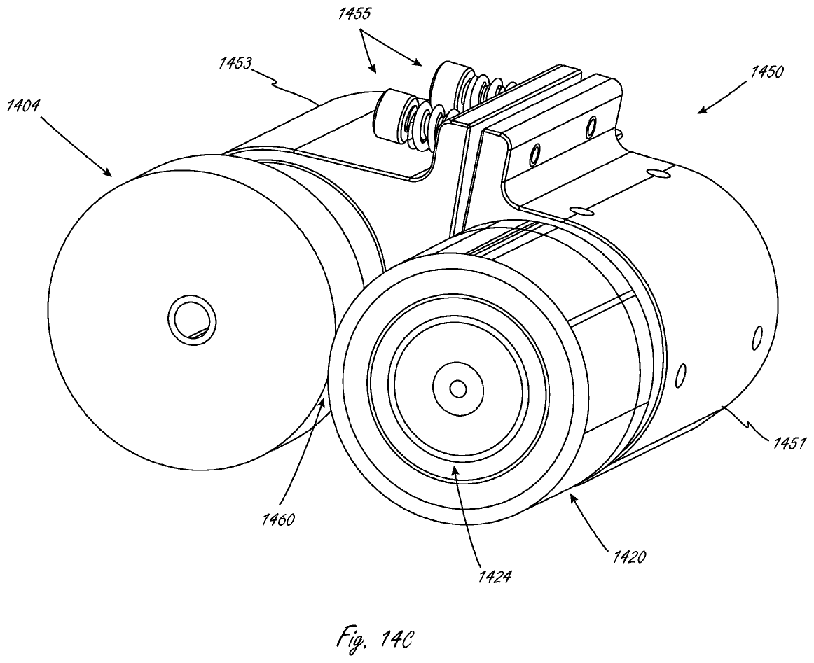

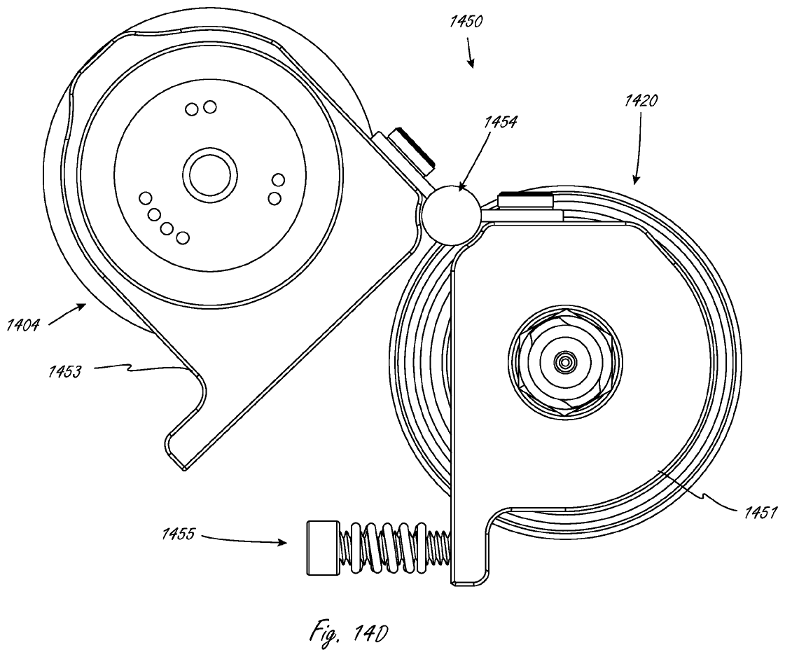

FIG. 14A is a perspective view of a portion of an exemplary blood pump system. Blood pump system 1400 includes motor 1402, which may include a stator and a rotor therein. Motor 1402 is rotationally coupled to mechanical driving member 1404, which is mechanically coupled to mechanical driven member 1420. Driving member 1404 and driven member 1420 are positioned to rotate around different axes, which is described in more detail herein. The blood pump system also includes magnetically coupled first and second magnetic assemblies, exemplary details of which are also described herein. The first magnetic assembly is coupled to mechanical driving member 1404, and a drive shaft is coupled to a second magnetic assembly that is rotated in response to rotation of the first magnetic assembly, exemplary details of which are described herein.

In this example, the driving member 1404 is mechanically coupled to driven member 1420, and in this example includes a friction drive or interface, which utilizes friction between the rotating cylindrical surface of driving member 1404 to cause the rotation of a cylindrical surface of driven member 1420. The mechanical driving and driven members 1404 and 1420 have, in this embodiment, cylindrical configurations in at least the regions where they interface and contact one another. In some mere examples, mechanical driving member 1404 may comprise a polyurethane material, although other materials may be used for the driving and driven members as long as rotation of driving member 1404 causes rotation of driven member 1420. Blood pump system 1400 also includes optional fluid inlets 1430′ and 1430″, which may be fluid inlets for a catheter clean purge fluid and a sheath clean fluid. In alternative designs, the system may include a single fluid inlet. The fluid inlets may be in fluid communication with catheter and sheath fluid pathways, examples of which are shown in FIG. 8B, for example. Blood pump system 1400 may include one or more outer housing components 1460 that provide one or more outer surfaces, and which house therein a portion of a return fluid pathway, which may in fluid communication with fluid outlet 1440, which may be in fluid communication with a waste reservoir.

In this example, as shown, and as is the case with the example in FIG. 13, the driving member interfaces with a reduced diameter portion of the outer magnetic assembly, which allows the driving member to have a desired diameter.

If a blood pump system includes a roller or friction drive (such as in the example in FIGS. 14A-14D), it may be beneficial to be able to at least one of control or maintain one or more of friction created when the driving member rotates (optionally including controlling or maintaining the amount of friction), contact between the driving and driven members (optionally including the amount of contact) and/or force applied to the driven member by the driving member (optionally including the amount of force). Any of these alone or in combination may influence the efficiency of the friction interface system, and may influence the performance of the blood pump. The example shown in FIGS. 14A-14D is a mere example of a particular non-limiting implementation of a blood pump system that is adapted such that at least one of friction between the driving and driven members (optionally including an amount of friction), contact between the rotating surfaces (optionally including an amount of contact), and/or the force applied to the driven member by the driving member (including an amount of force) may be controlled and/or maintained.

In this disclosure, adjusting or controlling the force may occur at any time before or during the procedure. In a merely exemplary use, the force may be adjusted (e.g., using tension adjusters) before the blood pump is inserted into the patient to help establish a desired pump performance.

In this disclosure, maintaining force and/or contact may occur at any time before or during the procedure, but it may be most useful during the procedure to ensure force/contact in the event or wear or simply to maintain a desired frictional force between the driving and driven members.

In the example of FIGS. 14A-14D, rotating the screw (part of the tensioning adjuster) may be part of adjusting the force (such as to establish a baseline setting), and the spring may help maintain force or contact between the driving and driven members during use by applying a force against the first portion 145 while the distal end of the screw is anchored in second portion 1451.

Blood pump system 1400 includes housing 1450 that includes a first member or portion 1453 and a second member or portion 1451. First member 1453 defines a lumen that is sized and configured to receive motor 1402 therein, as shown in FIG. 14A, wherein the motor rotates around first axis FA. Second member 1451 has a lumen therein that is sized and configured to receive therein one or more components that are secured to the one or more components that are adapted to rotate about the second axis SA, such as first and second magnetic assemblies (optionally outer and inner magnetic assemblies) and a drive shaft. In this example, an adaptor 1462 (FIG. 14B) is sized and configured to be disposed within second member 1451, as shown. Adaptor 1462 includes a lumen therein that is a portion of the return fluid pathway, as shown with the solid arrow (proximal is the right in the figure), and which is in communication with the fluid outlet 1440 (FIG. 14A). Adaptor 1462 is coupled to return fluid member 1426, as shown, which forms a part of the return fluid pathway, and which may be polymeric so as not to heat up in response to magnetic flux. Adaptor 1462 and return fluid member 1426 are not adapted to rotate, and both form part of the return fluid pathway.

In this merely exemplary example, at least one of the first and second members of the housing are adapted to move relative to the other about a pivot location 1454 (FIG. 14D), which may be part of a hinge. In this example, the blood pump system includes a force maintainer, which includes one or more tension adjusters 1455, which in this example each include a screw and a spring, as shown, which facilitate controllably adjusting the amount of contact, friction and/or force between the friction gear components, as well as maintaining force therebetween when in use. By controllably adjusting one or more of these parameters, the relative distance between the first and second axes may optionally also change.

An exemplary benefit of being able to adjust the amount of force (e.g., friction) between the driving and driven members is that, prior to use, a desired amount of force between the driving and driven members can be set to provide the desired pump performance including desired flow rate(s). For example the screw can be screwed further into the threaded apertures to increase a force between the driving and driven members. Additionally, it may be desirable to decrease the force if the force is too great and the motor is not efficiently causing the rotation of the impeller(s). In use, increasing contact and/or force may be used to maintain the force, such as within a desired range. It is thus understood that increasing a force as described herein may include maintaining a desired force, such as maintaining a force within a range or above a threshold.

An exemplary benefit of being able to maintain contact and/or force (e.g., friction) associated with the frictional interface between the driving and driven gears during use (without necessarily having to manually maintain) is that after a period of use, it may be necessary or maintain force (e.g. friction) or contact between the rotating surfaces. For example only, after some time, there may be some wear of one or more of the rotatable members, which may lead to less contact therebetween, and it may be desirable to maintain force and/or contact therebetween in the event of wear. For example, if one or more parts begin to wear down, the efficiency of the rotational coupling between the motor and impeller(s) may want to decrease, and it may be desirable to maintain force and/or contact to maintain a certain pump performance, for example. Additionally, the springs may simply maintain force between the driving and driven members even if wear is not expected to occur during use.

In this example, a force or contact maintainer may comprises a plurality of tension adjusters (e.g., 1455), each of which may include a spring and a screw, wherein the spring is adapted to maintain at least of force (e.g., optionally a constant force) on or contact with the driven member, even if the driving member and/or the driven member begin to wear during use. A spring constant may be chosen or selected to maintain force and/or contact. Maintaining force in this context does not require maintaining a constant force, but may refer generally to maintaining contact such that a force (e.g., friction) exists when rotated.

FIGS. 15A and 15B illustrate an exemplary blood pump system that may optionally in include any or all components from blood pump system 1400 herein. The blood pump system in FIGS. 15A and 15B optionally includes motor 1502 that is rotationally coupled to mechanical driving member 1504, which is mechanically coupled to mechanically driven member 1520. Mechanical driven member 1520 may be coupled to a first magnetic assembly, the rotation of which causes the rotation of a second magnetic assembly that is coupled to a drive shaft or other component in rotational communication with the drive shaft. Any other suitable feature from any other example herein may be incorporated into the blood pump system in FIGS. 15A and 15B.

The blood pump system in FIGS. 15A and 15B includes a case 1580 that includes a first portion 1582 and a second portion 1584, wherein the first and second portions are movable relative to one another (e.g., via a hinge) to provide access to a portion of a motor assembly. The case 1580 may act as an outer housing to the mechanical driving member 1504 and mechanical driven member 1520 therein. At least one of the first portion 1582 and second portion 1584 may include a housing receiving area that is sized and configured to stably receive a housing therein, such as housing 1250 or housing 1450, and in this example housing 1550 may optionally be the same as housing 1450 in any and all regards (e.g., first and second portions, at least one of which is movable relative to the another). The case 1580 may have one or more inner surfaces that are sized and configured to engage one or more non-rotatable members of the motor assembly such that the motor assembly sits within case 1580 such that any rotatable components (e.g., driving member 1504 or driven member 1520) do not contact inner surfaces of the case and interfere with their rotation. Additionally, the case 1580 can provide an outer housing and prevent nearby objects from engaging with the rotating components and interfere with the rotation thereof. Additionally, the case 1580 can allow the motor assembly to be placed on a surface of an object or a patient without interfering with the rotation of the mechanical driving and driven members, which may also provide heat protection from the motor.

In any of the examples herein, and which is shown generally in FIG. 14A, the assembly may include, between the motor and the first portion 1453, a deformable and/or flexible dampening member to dampen the vibration of the motor. In some examples, the dampening member includes a plurality of discrete annular or cylindrical rings of adjacent layers of material, each having a different stiffness. For example, the different layers of material may have different durometers. In a merely exemplary case, an outer layer (adjacent first portion 1453) may have a higher durometer material and an inner layer may have a lower durometer material. For example, an outer layer with a higher durometer (stiffer) may help seat the motor tightly within first portion 1453, and an inner layer may have a lower durometer to increase dampening. Any of the motor assemblies may include a plurality of different materials with different stiffnesses (e.g., durometers) as described herein. For example, the annular rings may include or consist of polyurethane.

Is it understood that any feature or aspect from any particular example or claim herein may be combined with any other suitably combinable feature or aspect of this disclosure, unless the disclosure herein indicates to the contrary.

1. A catheter blood pump, comprising:
a motor coupled to and in rotational communication with a mechanical driving member, the mechanical driving member rotatable about a first axis, the mechanical driving member mechanically coupled to a mechanical driven member rotatable about a second axis that is spaced from the first axis, wherein rotation of the mechanical driving member by the motor causes rotation of the mechanical driven member about the second axis,
the mechanical driven member coupled to a rotatable outer magnetic assembly that is rotatable about the second axis such that rotation of the mechanical driven member causes rotation of the outer magnetic assembly;
a rotatable inner magnetic assembly magnetically coupled with the outer magnetic assembly such that rotation of the outer magnetic assembly causes rotation of the inner magnetic assembly about the second axis;
a stationary return fluid member disposed around the rotatable inner magnetic assembly,
the inner magnetic assembly coupled with a proximal end of a drive shaft such that rotation of the inner magnetic assembly causes rotation of the drive shaft about the second axis, the drive shaft in rotational communication with a blood pump impeller;
a purge fluid inlet distal to the mechanical driven member, the purge inlet in communication with a clean purge pathway within a catheter; and
a return fluid outlet that is proximal to the mechanical driven member, the return fluid outlet in communication with a return fluid pathway,
the return fluid pathway including a lumen in the catheter and a volume created by and between the stationary return fluid member and the rotatable inner magnetic assembly.

2. The catheter blood pump of claim 1, wherein the rotatable outer magnetic assembly is coupled to an inner surface of the mechanical driven member.

3. The catheter blood pump of claim 1, wherein the rotatable outer magnetic assembly is coupled to an outer surface of the mechanical driven member.

4. The catheter blood pump of claim 1, wherein the mechanical coupling between the mechanical driving member and the mechanical driven member comprises a geared mechanical coupling.

5. The catheter blood pump of claim 4, wherein the geared mechanical coupling comprises a plurality of teeth on the mechanical driving member and a plurality of teeth on the mechanical driven member.

6. The catheter blood pump of claim 1, wherein the mechanical coupling comprises a friction drive between the mechanical driving member and the mechanical driven member.

7. The catheter blood pump of claim 1, further comprising a housing in which at least a portion of the motor is disposed, the housing sized and configured to maintain the first and second axes at a fixed distance.

8. The catheter blood pump of claim 1, further comprising a housing in which at least a portion of the motor is disposed, the housing sized and configured to maintain a force on the driven member by the driving member in the event of wear from at least one of the driving member or the driven member.

9. The catheter blood pump of claim 1, wherein the driving member has a diameter that is larger than a diameter of the driven member.

10. The catheter blood pump of claim 1, wherein the speed multiplication between the driving and driven member is greater than 1:1, optionally about 1.5:1, and optionally about 2:1.

11. The catheter blood pump of claim 1, wherein the driven member has an outer surface that interfaces with a surface of the driving member, the outer surface of the driven member has a smaller diameter than at least a portion of the outer magnetic assembly.

12. A catheter blood pump, comprising:
a motor coupled to and in rotational communication with a mechanical driving member, the mechanical driving member rotatable about a first axis, the mechanical driving member mechanically coupled to a mechanical driven member rotatable about a second axis that is spaced from the first axis, wherein rotation of the mechanical driving member by the motor causes rotation of the mechanical driven member about the second axis,
the mechanical driven member coupled to a rotatable outer magnetic assembly that is rotatable about the second axis such that rotation of the mechanical driven member causes rotation of the outer magnetic assembly;
a rotatable inner magnetic assembly magnetically coupled with the outer magnetic assembly such that rotation of the outer magnetic assembly causes rotation of the inner magnetic assembly about the second axis;
the inner magnetic assembly coupled with a proximal end of a drive shaft such that rotation of the inner magnetic assembly causes rotation of the drive shaft about the second axis, the drive shaft in rotational communication with a blood pump impeller;
a housing with a first portion and a second portion, the first portion sized and configured to receive at least a portion of the motor therein, the housing adapted to maintain at least one of force or contact on the driven member by the driving member.

13. The catheter blood pump of claim 12, wherein the housing includes a force maintainer that is adapted to maintain the at least one of force or contact.

14. The catheter blood pump of claim 13, wherein the force maintainer comprises a tension adjuster, the tension adjuster comprising a screw and a spring, wherein the first and second portions each include a threaded aperture therein to receive the screw, optionally wherein a spring constant helps maintain the at least one or force or contact.

15. The catheter blood pump of claim 12, further comprising a case with a first and second portions movable relative to each other to provide access to a housing receiving area in which the housing may be disposed, wherein when the first and second case portions are in a closed configuration, the mechanical driving and driven members are protected from contact from ambient objects.

16. The catheter blood pump of claim 12, wherein at least one of the first portion or the second portion is movable relative to the other.

17. The catheter blood pump of claim 16, wherein the housing includes a hinge that facilitates the relative motion between the first and second portion.

18. A catheter blood pump, comprising:
a motor coupled to and in rotational communication with a mechanical driving member, the mechanical driving member rotatable about a first axis, the mechanical driving member mechanically coupled to a mechanical driven member rotatable about a second axis that is spaced from the first axis, wherein rotation of the mechanical driving member by the motor causes rotation of the mechanical driven member about the second axis,
the mechanical driven member coupled to a rotatable outer magnetic assembly that is rotatable about the second axis such that rotation of the mechanical driven member causes rotation of the outer magnetic assembly;
a rotatable inner magnetic assembly magnetically coupled with the outer magnetic assembly such that rotation of the outer magnetic assembly causes rotation of the inner magnetic assembly about the second axis;
the inner magnetic assembly coupled with a proximal end of a drive shaft such that rotation of the inner magnetic assembly causes rotation of the drive shaft about the second axis, the drive shaft in rotational communication with a blood pump impeller;
a housing with a first portion and a second portion, the first portion sized and configured to receive at least a portion of the motor therein, at least one of the first portion and the second portion movable relative to the other, wherein the housing is adapted prevent the driving member from coming out of contact with the driven member during use and in the event of wear from at least one of the driving member or the driven member from use.

19. The catheter blood pump of claim 18, wherein at least one of the first portion and the second portion is movable about a hinge.

20. The catheter blood pump of claim 18, wherein one of the first axis and the second axis is movable, optionally rotatable, relative to the other.

21-29. (canceled)
